SEQUENCE LISTING (46 sequences)

SEQ ID NO: 1 - protein, 399 aa, Homo sapiens
MPQLSGGGGGGGGDPELCATDEMIPFKDEGDPQKEKIFAEISHPEEEGDLADIKSSLVNESEIIPASNGHEVARQAQTSQEPYHDKAREHPDDGKHPDGGLYNKGPSYSSYSGYIMMPNMNNDPYMSNGSLSPPIPRTSNKVPVVQPSHAVHPLTPLITYSDEHFSPGSHPSHIPSDVNSKQGMSRHPPAPDIPTFYPLSPGGVGQITPPLGWQGQPVYPITGGFRQPYPSSLSVDTSMSRFSHHMIPGPPGPHTTGIPHPAIVTPQVKQEHPHTDSDLMHVKPQHEQRKEQEPKRPHIKKPLNAFMLYMKEMRANVVAECTLKESAAINQILGRRWHALSREEQAKYYELARKERQLHMQLYPGWSARDNYGKKKKRKREKLQESASGTGPRMTAAYI

SEQ ID NO: 2 - protein, 384 aa, Homo sapiens
MPQLDSGGGGAGGGDDLGAPDELLAFQDEGEEQDDKSRDSAAGPERDLAELKSSLVNESEGAAGGAGIPGVPGAGAGARGEAEALGREHAAQRLFPDKLPEPLEDGLKAPECTSGMYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTGGKRNAFGTYPEKAAAPAPFLPMTVL

SEQ ID NO: 3 - protein, 588 aa, Homo sapiens
MPQLGGGGGGGGGGSGGGGGSSAGAAGGGDDLGANDELIPFQDEGGEEQEPSSDSASAQRDLDEVKSSLVNESENQSSSSDSEAERRPQPVRDTFQKPRDYFAEVRRPQDSAFFKGPPYPGYPFLMIPDLSSPYLSNGPLSPGGARTYLQMKWPLLDVPSSATVKDTRSPSPAHLSNKVPVVQHPHHMHPLTPLITYSNDHFSPGSPPTHLSPEIDPKTGIPRPPHPSELSPYYPLSPGAVGQIPHPLGWLVPQQGQPMYSLPPGGFRHPYPALAMNASMSSLVSSRFSPHMVAPAHPGLPTSGIPHPAIVSPIVKQEPAPPSLSPAVSVKSPVTVKKEEEKKPHVKKPLNAFMLYMKEMRAKVVAECTLKESAAINQILGRKWHNLSREEQAKYYELARKERQLHSQLYPTWSARDNYGKKKKRKREKQLSQTQSQQQVQEAEGALASKSKKPCVQYLPPEKPCDSPASSHGSMLDSPATPSAALASPAAPAATHSEQAQPLSLTTKPETRAQLALHSAAFLSAKAAASSSGQMGSQPPLLSRPLPLGSMPTALLASPPSFPATLHAHQALPVLQAQPLSLVTKSAH

SEQ ID NO: 4 - protein, 602 aa, Homo sapiens
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTLHFQSGSTHYSAYKTIEHQIAVQYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNDANTPKKCRALFGLDRQTLWCKPCRRKKKCVRYIQGEGSCLSPPSSDGSLLDSPPPSPNLLGSPPRDAKSQTEQTQPLSLSLKPDPLAHLSMMPPPPALLLAEATHKASALCPNGALDLPPAALQPAAPSSSIAQPSTSSLHSHSSLAGTQPQPLSLVTKSLE

SEQ ID NO: 5 - protein, 779 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTD

SEQ ID NO: 6 - protein, 397 aa, Mus musculus
MPQLSGGGGGGDPELCATDEMIPFKDEGDPQKEKIFAEISHPEEEGDLADIKSSLVNESEIIPASNGHEVVRQAPSSQEPYHDKAREHPDEGKHPDGGLYNKGPSYSSYSGYIMMPNMNSDPYMSNGSLSPPIPRTSNKVPVVQPSHAVHPLTPLITYSDEHFSPGSHPSHIPSDVNSKQGMSRHPPAPEIPTFYPLSPGGVGQITPPIGWQGQPVYPITGGFRQPYPSSLSGDTSMSRFSHHMIPGPPGPHTTGIPHPAIVTPQVKQEHPHTDSDLMHVKPQHEQRKEQEPKRPHIKKPLNAFMLYMKEMRANVVAECTLKESAAINQILGRRWHALSREEQAKYYELARKERQLHMQLYPGWSARDNYGKKKKRKREKLQESTSGTGPRMTAAYI

SEQ ID NO: 7 - protein, 303 aa, Mus musculus
MYKETVYSAFNLLMPYPPASGAGQHPQPQPPLHNKPGQPPHGVPQLSPLYEHFSSPHPTPAPADISQKQGVHRPLQTPDLSGFYSLTSGSMGQLPHTVSWPSPPLYPLSPSCGYRQHFPAPTAAPGAPYPRFTHPSLMLGSGVPGHPAAIPHPAIVPSSGKQELQPYDRNLKTQAEPKAEKEAKKPVIKKPLNAFMLYMKEMRAKVIAECTLKESAAINQILGRRWHALSREEQAKYYELARKERQLHMQLYPGWSARDNYGKKKRRSREKHQESTTGGKRNAFGTYPEKAAAPAPFLPMTVL

SEQ ID NO: 8 - protein, 599 aa, Mus musculus
MPQLGGGRGGGAGGGGGGSGAGATSGGDDLGANDELIPFQDEGGEEQEPSSDSASAQRDLDEVKSSLVNESENQSSSSDSEAERRPQPARDAFQKPRDYFAEVRRPQDGAFFKGPAYPGYPFLMIPDLSSPYLSNGPLSPGGARTYLQMKWPLLDVPSSATVKDTRSPSPAHLYGDPARWMVPPTFRSNKVPVVQHPHHMHPLTPLITYSNDHFSPASPPTHLSPEIDPKTGIPRPPHPSELSPYYPLSPGAVGQIPHPLGWLVPQQGQPMYSLPPGGFRHPYPALAMNASMSSLVSSRFPHMVAPAHPGLPTSGIPHPAIVSPIVKQEPAAPSLSPAVSAKSPVTVKKEEEKKPHVKKPLNAFMLYMKEMRAKVVAECTLKESAAINQILGRKWHNLSREEQAKYYELARKERQLHAQLYPTWSARDNYGKKKKRKREKQLSQTQSQQQIQEAEGALASKSKKPCIQYLPPEKPCDSPASSHGSTLDSPATPSAALASPAAPAATHSEQAQPLSLTTKPEARAQLALHSAAFLSAKAAASNSSQMGSQPPLLSRPLPLGSMPAALLTSPPTFPATLHAHQALPVLQAQPLSLVTKSAH

SEQ ID NO: 9 - protein, 459 aa, Mus musculus
MPQLNGGGGDDLGANDELISFKDEGEQEEKNSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQTLKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASMSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCSL

SEQ ID NO: 10 - protein, 781 aa, Mus musculus
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEALGYRQDDPSYRSFHSGGYGQDALGMDPMMEHEMGGHHPGADYPVDGLPDLGHAQDLMDGLPPGDSNQLAWFDTDL

SEQ ID NO: 11 - protein, 483 aa, Homo sapiens
MSGGGPSGGGPGGSGRARTSSFAEPGGGGGGGGGGPGGSASGPGGTGGGKASVGAMGGGVGASSSGGGPGGSGGGGSGGPGAGTSFPPPGVKLGRDSGKVTTVVATLGQGPERSQEVAYTDIKVIGNGSFGVVYQARLAETRELVAIKKVLQDKRFKNRELQIMRKLDHCNIVRLRYFFYSSGEKKDELYLNLVLEYVPETVYRVARHFTKAKLTIPILYVKVYMYQLFRSLAYIHSQGVCHRDIKPQNLLVDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFKSRTPPEAIALCSSLLEYTPSSRLSPLEACAHSFFDELRCLGTQLPNNRPLPPLFNFSAGELSIQPSLNAILIPPHLRSPAGTTTLTPSSQALTETPTSSDWQSTDATPTLTNSS

SEQ ID NO: 12 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 13 - protein, 490 aa, Mus musculus
MSGGGPSGGGPGGSGRARTSSFAEPGGGGGGGGGGPGGSASGPGGTGGGKASVGAMGGGVGASSSGGGPSGSGGGGSGGPGAGTSFPPPGVKLGRDSGKVTTVVATVGQGPERSQEVAYTDIKVIGNGSFGVVYQARLAETRELVAIKKVLQDKRFKNRELQIMRKLDHCNIVRLRYFFYSSGEKKDELYLNLVLEYVPETVYRVARHFTKAKLITPIIYIKVYMYQLFRSLAYIHSQGVCHRDIKPQNLLVDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFKSSKTPPEAIALCSSLLEYTPSSRLSPLEACAHSFFDELRRLGAQLPNDRPLPPLFNFSPGELSIQPSLNAILIPPHLRSPAGPASPLTTSYNPSSQALTEAQTGQDWQPSDATTATLASSS

SEQ ID NO: 14 - protein, 420 aa, Mus musculus
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASPPANATAASDTNAGDRGQTNNAASASASNST

SEQ ID NO: 15 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 16 - protein, 352 aa, Mus musculus
MAPLGYLLVLCSLKQALGSYPIWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 17 - DNA, 21 bp, Artificial Sequence
  Synthetic
gccacagaaatttgctgaaga

SEQ ID NO: 18 - DNA, 21 bp, Artificial Sequence
  Synthetic
ggtttgcttgtaatgggttca

SEQ ID NO: 19 - DNA, 29 bp, Artificial Sequence
  Synthetic
ataacgcgtccagcgtggcaatggctcga

SEQ ID NO: 20 - DNA, 33 bp, Artificial Sequence
  Synthetic
tgtcgtccaccacaagaatttaacatttgtttt

SEQ ID NO: 21 - DNA, 33 bp, Artificial Sequence
  Synthetic
ttcttgtggtggacgacagactgccttcaaatt

SEQ ID NO: 22 - DNA, 31 bp, Artificial Sequence
  Synthetic
atagcggccgcttacttgtcatcgtcgtcct

SEQ ID NO: 23 - DNA, 29 bp, Artificial Sequence
  Synthetic
ataacgcgtccagcgtggcaatggctcga

SEQ ID NO: 24 - DNA, 31 bp, Artificial Sequence; identical to SEQ ID NO: 22
  Synthetic

SEQ ID NO: 25 - DNA, 22 bp, Artificial Sequence
  Synthetic
gaagcagaagaggatcaccttg

SEQ ID NO: 26 - DNA, 21 bp, Artificial Sequence
  Synthetic
ttcttaaggctgagctgcaag

SEQ ID NO: 27 - DNA, 20 bp, Artificial Sequence
  Synthetic
tcactgtgctgcctccaagt

SEQ ID NO: 28 - DNA, 18 bp, Artificial Sequence
  Synthetic
gggcactgatccgcaaac

SEQ ID NO: 29 - DNA, 20 bp, Artificial Sequence
  Synthetic
tccaggccatcaagagtttc

SEQ ID NO: 30 - DNA, 20 bp, Artificial Sequence
  Synthetic
atctgctgggttctccactg

SEQ ID NO: 31 - DNA, 16 bp, Artificial Sequence
  Synthetic
gcagcccacgggtcaa

SEQ ID NO: 32 - DNA, 18 bp, Artificial Sequence
  Synthetic
cggttgctcggactgctt

SEQ ID NO: 33 - DNA, 20 bp, Artificial Sequence
  Synthetic
ttccgagctagcttttgagg

SEQ ID NO: 34 - DNA, 20 bp, Artificial Sequence
  Synthetic
acaccggggtttagggttag

SEQ ID NO: 35 - DNA, 19 bp, Artificial Sequence
  Synthetic
tgaagcaggcatctgaggg

SEQ ID NO: 36 - DNA, 20 bp, Artificial Sequence
  Synthetic
cgaaggtggaagagtgggag

SEQ ID NO: 37 - DNA, 20 bp, Artificial Sequence
  Synthetic
tccagaagagggcgtcagat

SEQ ID NO: 38 - DNA, 20 bp, Artificial Sequence
  Synthetic
caaatcccagcaaccacatg

SEQ ID NO: 39 - DNA, 20 bp, Artificial Sequence
  Synthetic
ttaggtgtctgccagcctct

SEQ ID NO: 40 - DNA, 20 bp, Artificial Sequence
  Synthetic
aaccaggtgcagtggatagg

SEQ ID NO: 41 - DNA, 20 bp, Artificial Sequence
  Synthetic
gggggaaaacacagcttaca

SEQ ID NO: 42 - DNA, 20 bp, Artificial Sequence
  Synthetic
ttgactgggtcgcttctctt

SEQ ID NO: 43 - protein, 862 aa, Homo sapiens
MNIQEQGFPLDLGASFTEDAPRPPVPGEEGELVSTDPRPASYSFCSGKGVGIKGETSTATPRRSDLDLGYEPEGSASPTPPYLKWAESLHSLLDDQDGISLFRTFLKQEGCADLLDFWFACTGFRKLEPCDSNEEKRLKLARAIYRKYILDNNGIVSRQTKPATKSFIKGCIMKQLIDPAMFDQAQTEIQATMEENTYPSFLKSDIYLEYTRTGSESPKVCSDQSSGSGTGKGISGYLPTLNEDEEWKCDQDMDEDDGRDAAPPGRLPQKLLLETAAPRVSSSRRYSEGREFRYGSWREPVNPYYVNAGYALAPATSANDSEQQSLSSDADTLSLTDSSVDGIPPYRIRKQHRREMQESVQVNGRVPLPHIPRTYRVPKEVRVEPQKFAEELIHRLEAVQRTREAEEKLEERLKRVRMEEEGEDGDPSSGPPGPCHKLPPAPAWHHFPPRCVDMGCAGLRDAHEENPESILDEHVQRVLRTPGRQSPGPGHRSPDSGHVAKMPVALGGAASGHGKHVPKSGAKLDAAGLHHHRHVHHHVHHSTARPKEQVEAEATRRAQSSFAWGLEPHSHGARSRGYSESVGAAPNASDGLAHSGKVGVACKRNAKKAESGKSASTEVPGASEDAEKNQKIMQWIIEGEKEISRHRRTGHGSSGTRKPQPHENSRPLSLEHPWAGPQLRTSVQPSHLFIQDPTMPPHPAPNPLTQLEEARRRLEEEEKRASRAPSKQRYVQEVMRRGRACVRPACAPVLHVVPAVSDMELSETETRSQRKVGGGSAQPCDSIVVAYYFCGEPIPYRTLVRGRAVTLGQFKELLTKKGSYRYYFKKVSDEFDCGVVFEEVREDEAVLPVFEEKIIGKVEKVD

SEQ ID NO: 44 - protein, 843 aa, Homo sapiens
MSSAMLVTCLPDPSSSFREDAPRPPVPGEEGETPPCQPGVGKGQVTKPMPVSSNTRRNEDGLGEPEGRASPDSPLTRWTKSLHSLLGDQDGAYLFRTFLEREKCVDTLDFWFACNGFRQMNLKDTKTLRVAKAIYKRYIENNSIVSKQLKPATKTYIRDGIKKQQIDSIMFDQAQTEIQSVMEENAYQMFLTSDIYLEYVRSGGENTAYMSNGGLGSLKVVCGYLPTLNEEEEWTCADFKCKLSPTVVGLSSKTLRATASVRSTETVDSGYRSFKRSDPVNPYHIGSGYVFAPATSANDSEISSDALTDDSMSMTDSSVDGIPPYRVGSKKQLQREMHRSVKANGQVSLPHFPRTHRLPKEMTPVEPATFAAELISRLEKLKLELESRHSLEERLQQIREDEEREGSELTLNSREGAPTQHPLSLLPSGSYEEDPQTILDDHLSRVLKTPGCQSPGVGRYSPRSRSPDHHHHHHSQYHSLLPPGGKLPPAAASPGACPLLGGKGFVTKQTTKHVHHHYIHHHAVPKTKEEIEAEATQRVHCFCPGGSEYYCYSKCKSHSKAPETMPSEQFGGSRGSTLPKRNGKGTEPGLALPAREGGAPGGAGALQLPREEGDRSQDVWQWMLESERQSKPKPHSAQSTKKAYPLESARSSPGERASRHHLWGGNSGHPRTTPRAHLFTQDPAMPPLTPPNTLAQLEEACRRLAEVSKPPKQRCCVASQQRDRNHSATVQTGATPFSNPSLAPEDHKEPKKLAGVHALQASELVVTYFFCGEEIPYRRMLKAQSLTLGHFKEQLSKKGNYRYYFKKASDEFACGAVFEEIWEDETVLPMYEGRILGKVERID

SEQ ID NO: 45 - protein, 868 aa, Mus musculus
MQSPKMNVQEQGFPLDLGASFTEDAPRPPVPGEEGELVSTDSRPVNHSFCSGKGTSIKSETSTATPRRSDLDLGYEPEGSASPTPPYLRWAESLHSLLDDQDGISLFRTFLKQEGCADLLDFWFACSGFRKLEPCDSNEEKRLKLARAIYRKYILDSNGIVSRQTKPATKSFIKDCVMKQQIDPAMFDQAQTEIQSTMEENTYPSFLKSDIYLEYTRTGSESPKVCSDQSSGSGTGKGMSGYLPTLNEDEEWKCDQDADEDDGRDPLPPSRLTQKLLLETAAPRAPSSRRYNEGRELRYGSWREPVNPYYVNSGYALAPATSANDSEQQSLSSDADTLSLTDSSVDGIPPYRIRKQHRREMQESIQVNGRVPLPHIPRTYRMPKEIRVEPQKFAEELIHRLEAVQRTREAEEKLEERLKRVRMEEEGEDGEMPSGPMASHKLPSVPAWHHFPPRYVDMGCSGLRDAHEENPESILDEHVQRVMRTPGCQSPGPGHRSPDSGHVAKTAVLGGTASGHGKHVPKLGLKLDTAGLHHHRHVHHHVHHNSARPKEQMEAEVARRVQSSFSWGPETHGHAKPRSYSENAGTTLSAGDLAFGGKTSAPSKRNTKKAESGKNANAEVPSTTEDAEKNQKIMQWIIEGEKEISRHRKAGHGSSGLRKQQAHESSRPLSIERPGAVHPWVSAQLRNSVQPSHLFIQDPTMPPNPAPNPLTQLEEARRRLEEEEKRANKLPSKQRYVQAVMQRGRTCVRPACAPVLSVVPAVSDLELSETETKSQRKAGGGSAPPCDSIVVAYYFCGEPIPYRTLVRGRAVTLGQFKELLTKKGSYRYYFKKVSDEFDCGVVFEEVREDEAVLPVFEEKIIGKVEKVD

SEQ ID NO: 46 - protein, 840 aa, Mus musculus
MSSAVLVTLLPDPSSSFREDAPRPPVPGEEGETPPCQPSVGKVQSTKPMPVSSNARRNEDGLGEPEGRASPDSPLTRWTKSLHSLLGDQDGAYLFRTFLEREKCVDTLDFWFACNGFRQMNLKDTKTLRVAKAIYKRYIENNSVVSKQLKPATKTYIRDGIKKQQIGSVMFDQAQTEIQAVMEENAYQVFLTSDIYLEYVRSGGENTAYMSNGGLGSLKVLCGYLPTLNEEEEWTCADLKCKLSPTVVGLSSKTLRATASVRSTETAENGFRSFKRSDPVNPYHVGSGYVFAPATSANDSELSSDALTDDSMSMTDSSVDGVPPYRMGSKKQLQREMHRSVKANGQVSLPHFPRTHRLPKEMTPVEPAAFAAELISRLEKLKLELESRHSLEERLQQIREDEEKEGSEQALSSRDGAPVQHPLALLPSGSYEEDPQTILDDHLSRVLKTPGCQSPGVGRYSPRSRSPDHHHQHHHHQQCHTLLPTGGKLPPVAACPLLGGKSFLTKQTTKHVHHHYIHHHAVPKTKEEIEAEATQRVRCLCPGGTDYYCYSKCKSHPKAPEPLPGEQFCGSRGGTLPKRNAKGTEPGLALSARDGGMSSAAGAPQLPGEEGDRSQDVWQWMLESERQSKSKPHSAQSIRKSYPLESACAAPGERVSRHHLLGASGHSRSVARAHPFTQDPAMPPLTPPNTLAQLEEACRRLAEVSKPQKQRCCVASQQRDRNHSAAGQAGASPFANPSLAPEDHKEPKKLASVHALQASELVVTYFFCGEEIPYRRMLKAQSLTLGHFKEQLSKKGNYRYYFKKASDEFACGAVFEEIWDDETVLPMYEGRILGKVERID

(19) United States
(12) Patent Application Publication
(10) Pub. No.: US 2013/0273649 A1
(43) Pub. Date: Oct. 17, 2013

(54) Culture medium for pluripotent stem cells

(71) Applicant: UNIVERSITY OF SOUTHERN CALIFORNIA, Los Angeles, CA (US)

(72) Inventors: Jun Wu, Alhambra, CA (US); QiLong Ying, Arcadia, CA (US); Hoon Kim, Los Angeles, CA (US)

(73) Assignee: University of Southern California, Los Angeles, CA (US)

(21) Appl. No.: 13/861,477

(22) Filed: Apr. 12, 2013

Related U.S. Application Data
(60) Provisional application No. 61/623,717, filed on Apr. 13, 2012.

(51) Int. Cl. C12N 5/0735

(52) U.S. Cl. CPC C12N 5/0606
USPC 435/349; 435/377; 435/354; 435/353; 435/352; 435/366; 435/325

(57) ABSTRACT

The present invention provides culture media and methods of culturing pluripotent stem cells, such as epiblast stem cells (EpiSCs) and embryonic stem cells (ESCs), in order to culture, derive, and reprogram pluripotent stem cells, such as converting ESCs to EpiSCs.

### CROSS-REFERENCE

This application claims priority to U.S. Provisional Patent Application Ser. No. 61/623,717 filed Apr. 13, 2012, incorporated by reference herein it its entirety.

### BACKGROUND OF THE INVENTION

While advances have been made in maintaining stem cells in culture, the use of feeder cells and/or feeder cell extracts is a common requirement for all pluripotent stem cell cultures. Since feeders are derived from fetal tissue, they are heterogeneous from batch to batch and range in quality by strain and handling.

### SUMMARY OF THE INVENTION

In a first aspect, the present invention provides compositions, comprising

(a) a compound that can stabilize axin; and

(b) a compound that can stabilize β-catenin.

In one embodiment, the compositions comprise

(a) an inhibitor of β-catenin binding to T-cell factors (Tcfs); and

(b) a suppressor of glycogen synthase kinase (GSK3) activation.

In a further embodiment, the inhibitor of β-catenin binding to Tcfs is selected from the group consisting of 3,5,7,8-Tetrahydro-2-[4-(trifluoromethyl)phenyl]-4H-thiopyrano[4,3-d]pyrimidin-4-one (XAV939), 4-(1,3,3a,4,7,7a-Hexahydro-1,3-dioxo-4,7-methano-2H-isoindol-2-yl)-N-8-quinolinyl-Benzamide (IWR-1), and (1R,4r)-4-((2s,3aR,4R,7S,7aS)-1,3-dioxooctahydro-1H-4,7-methanoinden-2-yl)-N-(quinolin-8-yl)cyclohexanecarboxamide (53AH), or salts thereof. In another embodiment, the suppressor of GSK3 activation is selected from the group consisting of 6-((2-((4-(2,4-Dichlorophenyl)-5-(4-methyl-1H-imidazol-2-yl)pyrimidin-2-yl)amino)ethyl)amino)nicotinonitrile (CHIR99021), 2,6-Pyridinediamine, N6-[2-[[4-(2,4-dichlorophenyl)-5-(1H-imidazol-1-yl)-2-pyrimidinyl]amino]ethyl]-3-nitro- (CHIR 98014), benzyl-2-methyl-1,2,4-thiadiazolidine-3,5-dione (TDZD-8), 3-(2,4-Dichlorophenyl)-4-(1-methyl-1H-indol-3-yl)-1H-pyrrole-2,5-dione (SB216763), 3-[(3-Chloro-4-hydroxyphenyl)amino]-4-(2-nitrophenyl)-1H-pyrrol-2,5-dione (SB415286); 2,6-Pyridinediamine, N6-[2-[[4-(2,4-dichlorophenyl)-5-(1H-imidazol-1-yl)-2-pyrimidinyl]amino]ethyl]-3-nitro-; N-[(4-Methoxyphenyl)methyl]-N′-(5-nitro-2-thiazolyl); and Wnt3a (SEQ ID NO: 15 or 16), or salts thereof.

In a further embodiment, the compositions are present in a cell culture medium.

In another aspect, the present invention provides methods for culturing pluripotent stem cells, comprising culturing the pluripotent stem cells in the culture medium of any embodiment of the invention, under conditions suitable for culturing the pluripotent stem cells. In one embodiment, the pluripotent stem cells comprise embryonic stem cells (ESCs) or epiblast-derived stem cells (EpiSCs).

In another aspect, the present invention provided methods for generating a pluripotent cell line from a tissue, comprising

(a) culturing a tissue comprising a pluripotent cell in a cell culture medium of any embodiment of the invention; and

(b) isolating the pluripotent cells in the culture medium.

### DESCRIPTION OF THE FIGURES

FIG. 1. CHIR/XAV Supports Self-Renewal and de novo Derivation of Mouse EpiSCs

(A) Representative phase-contrast images of CD1 mouse EpiSCs cultured in the indicated conditions. Mouse EpiSCs cultured in FGF2/activin remained undifferentiated (left panel). They differentiated 3 days after the removal of FGF2/activin and the addition of 3 μM CHIR99021 (middle panel) or 2i (3 μM CHIR99021+1 μM PD0325901) (right panel). (B) Phase-contrast image showing differentiating CD1 mouse EpiSCs at the 2nd passage in GMEM/10% FBS with 2 μM XAV. (C) CD1 mouse EpiSCs were cultured in CHIR/XAV for 7 passages and were subsequently immunostained with indicated antibodies against pluripotency markers. (D) Numbers of undifferentiated colonies formed by day 9 after single-cell deposition of CD1 EpiSCs into 0.1% gelatin-coated 96-well plates and cultured in the conventional mouse ESC medium supplemented with the indicated small molecules or cytokines Results are shown as mean±s.d. of six biological replicates. (E) De novo derivation of mouse EpiSCs. Epiblast tissue (outlined in dashed line) of the E5.75 CD1 mouse embryo was dissected and cultured in CHIR/XAV. The outgrowth formed from the plated epiblast (middle panel) was disaggregated to establish a stable EpiSC line (right panel). Thirteen EpiSC lines were established from 13 plated CD1 mouse embryos. Scale bars, 50 μm (A, B, C and E). See also FIG. 8.

FIG. 2. Molecular Signatures and Pluripotency of EpiSCs Derived and Maintained in CHIR/XAV

(A) qRT-PCR analysis of gene expression in mouse EpiSCs maintained in CHIR/XAV or FGF2/activin. Expression levels are relative to those of mouse ESCs maintained in 2i. Data represent mean±s.d of triplicate samples from three independent experiments. (B) Bisulfite sequencing of DNA methylation of the promoter regions of Stella, Oct4, and Vasa in mouse ESCs maintained in 2i and EpiSCs maintained in CHIR/XAV. (C) Quantification of Oct4 distal enhancer (DE) and proximal enhancer (PE) reporter activities in mouse ESCs and EpiSCs. Data represent mean±s.d. of three experimental replicates. (D) Immunostaining showing Tuj-positive neurons (ectoderm), myosin-positive beating cardiomyocytes (mesoderm), and Gata4-positive endoderm cells derived from CD1 mouse EpiSCs through EB formation. (E) Hematoxylin and eosin (H&E) staining of teratomas generated from CD1 mouse EpiSCs derived and cultured in CHIR/XAV. Scale bars, 50 μm (D and E).

FIG. 3. Mouse EpiSCs Maintained in CHIR/XAV Represent Early-Stage Epiblast Cells

(A) Heatmap of global gene expression patterns in mouse ESCs and EpiSCs. Of the total number of genes, 9.39% show more than a 1.5-fold difference in gene expression levels between mouse ESCs and the two groups of EpiSCs. Intensity plot is shown at the bottom. (B) Scatter plot analyses comparing global gene expression patterns among the three groups of cells. The r2 value (square of linear correlation) in each plot was obtained by comparing global gene expression (35,556 transcripts total) in the two indicated samples. (C-E) Phase contrast and fluorescence images of purified Oct4-GFP-positive EpiSCs after 7 passages in FGF2/activin (C), 7 passages in CHIR/XAV (D), or 21 passages in CHIR/XAV (E). Representative flow cytometry analyses of Oct4-GFP expression are shown in the bottom panels. Scale bars, 50 μm.

FIG. 4. Stabilization of Axin2 Mediates Self-Renewal of EpiSCs Maintained in CHIR/XAV or CHIR/IWR-1

(A) TOPFlash™ assay in CD1 mouse EpiSCs treated with the indicated inhibitors for 12 hours. Data represent mean±s.d. of three biological replicates. (B) Representative phase contrast images of CD1 mouse EpiSCs cultured in the indicated conditions for 7 days. (C) Western blot analysis of CD1 mouse EpiSCs treated with the indicated inhibitors for 12 h. (D) qRT-PCR analysis of Axin1 and Axin2 mRNA levels in CD1 mouse EpiSCs stably transfected with Axin1 shRNA or Axin2 shRNA. Data represent mean±s.d. of three biological replicates. (E) Colony assay on CD1 mouse EpiSCs stably transfected with scramble, Axin1, or Axin2 shRNAs. Cells were plated onto a 6-well plate at a density of 5,000 cells/well and cultured in CHIR/IWR-1. Colonies were counted 7 days after plating. Data represent the total combined numbers of colonies from two independent experiments in which each shRNA-transfected group was cultured in one well of a 6-well plate. (F) Representative phase contrast images of CD 1 mouse EpiSCs stably transfected with the indicated shRNAs and cultured in CHIR/IWR-1 for 7 days. (G) Western blot analysis of CD1 mouse EpiSCs overexpressing Flag-tagged Axin1 or Axin2. (H) Representative phase contrast images of CD1 mouse EpiSCs overexpressing Axin1 or Axin2 and cultured in CHIR for 7 days. Axin2-overexpressing EpiSCs could be continually passaged in CHIR alone. Scale bars, 50 μm (B, F and H)

FIG. 5. Axin2-Mediated EpiSC Self-Renewal is 13-Catenin-Dependent

(A) Locus map of the mouse genome with loxP sites located in introns 1 and 6 of the Ctnnb 1 (β-catenin) gene. Expression of Cre recombinase excises exons 2 to 6. (B) Immunocytochemistry showing strong Oct4 staining in most β-catenin^{fl/fl} EpiSCs derived and maintained in FGF2/activin. (C) Western blot analysis confirming the loss of β-catenin in β-catenin^{-/-} EpiSCs. (D) TOPFlash™ assay in β-catenin^{fl/dl} and β-catenin^{-/-} EpiSCs treated with 3 μM CHIR for 12 h. Data represent mean±s.d. of three biological replicates. (E) Immunocytochemistry showing strong Oct4 staining in most β-catenin^{-/-} EpiSCs maintained in FGF2/activin. (F) Representative image of β-catenin^{-/-} EpiSCs cultured in CHIR/XAV for 7 days after the removal of FGF2/activin. In the absence of FGF2/activin, β-catenin^{-/-} EpiSCs cultured in basal medium (GMEM/10% FBS) or basal medium supplemented with CHIR/XAV or CHIR/IWR-1 differentiated and could not be maintained beyond passage 2 or 3. (G) Western blot analysis of β-catenin^{-/-} EpiSCs overexpressing Flag-tagged Axin2. β-catenin^{-/-} EpiSCs transfected with an empty vector were used as a control. (H) Representative phase contrast image of β-catenin^{-/-} EpiSCs overexpressing Flag-tagged Axin2 and cultured in basal medium only (No treatment) or basal medium plus 3 μM CHIR for 7 days after the removal of FGF2/activin. Scale bars, 50 μm (B, E, F and H).

FIG. 6. Retention of Stabilized 13-Catenin in the Cytoplasm Maintains EpiSC Self-Renewal

(A) Western blot analysis of cytoplasmic, nuclear and total β-catenin levels in CD1 mouse EpiSCs overexpressing Flag-tagged Axin1 or Axin2. Cells were either untreated or treated with 3 μM CHIR for 12 h. (B) Immunostaining of CD1 mouse EpiSCs overexpressing Flag-tagged Axin2 (C) Co-IP of Flag or β-catenin in CD1 mouse EpiSCs overexpressing empty vector or Flag-tagged Axin2. Cells were treated with 3 μM CHIR for 12 h (D) Immunostaining of ΔNβ-catenin-ERT2-overexpressing CD1 mouse EpiSCs before and after treatment with 1 μM 4-OHT. (E). Western blot analysis of cytoplasmic and nuclear ΔNβ-catenin-ERT2 levels after treatment with 1 μM 4-OHT for 24 h. (F). Phase contrast image of ΔNβ-catenin-ERT2-EpiSCs after 25 passages in basal medium only. (G) qRT-PCR analysis of Oct4, Nanog and Fgf5 mRNA levels in ΔNβ-catenin-ERT2-EpiSCs maintained in basal medium or basal medium plus FGF2/activin for 11 passages. Data represent mean±s.d. of three biological replicates. (H) Phase contrast image of ΔNβ-catenin-ERT2-EpiSCs after treatment with 1 μM 4-OHT for 24 h. (I) Phase contrast and fluorescent images of floxed ΔNβ-catenin-ERT2-EpiSCs cultured in the indicated conditions for 7 days after Cre-recombinase-mediated excision of the ΔNβ-catenin-ERT2 transgene. GFP expression was driven by the constitutive CAG promoter after excision of the floxed ΔNβ-catenin-ERT2-STOP cassette. Scale bars, 50 μm (B, D, F, H and I).

See also FIG. 9.

FIG. 7: β-Catenin Mediates Human ESC Self-Renewal Through a Mechanism Similar to that in Mouse EpiSCs.

(A) TOPFlash™ assay in H9 human ESCs subjected to the indicated treatments for 24 h. Data represent mean±s.d. of three biological replicates. (B) Representative phase contrast and alkaline phosphatase (AP) staining images of H9 human ESCs cultured in the indicated conditions for 3 passages. (C) Phase contrast images of H9 human ESCs cultured in CHIR/XAV or CHIR/IWR-1 for 11 passages. (D) Colony forming efficiency assay of H9 human ESCs cultured in FGF2 or CHIR/IWR-1 conditions. Data represent mean±s.d. of three biological replicates. Right panel: a representative image showing AP staining of colonies formed from H9 human ESCs cultured in either FGF2 or CHIR/IWR-1 condition. (E) Human ESCs cultured in CHIR/IWR-1 for 11 passages were immunostained with the indicated antibodies. (F) Embryoid bodies (EBs) were generated from H9 human ESCs cultured in CHIR/IWR-1 for 11 passages. The outgrowths of EBs were immunostained with the indicated antibodies. (G) H&E staining of teratomas generated from H9 ESCs cultured in CHIR/IWR-1 for 20 passages. (H) Western blot analysis of Axin1 and Axin2 expression in HES3 human ESCs treated with the indicated cytokines/inhibitors for 24 h. (I) Immunofluorescence images of H9 human ESCs (passage 5 in CHIR) overexpressing Flag-tagged Axin2. (J) Representative phase contrast and immunofluorescence images of HES2 human ESCs overexpressing ΔNβ-catenin-ERT2 at passage 5 in basal medium only. These cells were cultured in basal medium only for more than 10 passages and remained morphologically undifferentiated. (K) Phase contrast images of HES2 human ESCs overexpressing ΔNβ-catenin (left panel, passage 2 in basal medium/FGF2) or ΔNβ-catenin/A295W/I296W mutant (right panel, passage 5 in basal medium only). Scale bars, 50 μm (B, C, E-G, and I-K). (L) Model of mouse EpiSC and human ESC self-renewal mediated by β-catenin. In the absence of Wnt or GSK3 inhibitor, a β-catenin destruction complex, containing Axin1, GSK3, and APC is formed, leading to the degradation of β-catenin and differentiation (left). In the presence of Wnt or GSK3 inhibitor, β-catenin is stabilized and can initiate cellular responses related to both self-renewal and differentiation. Stabilized β-catenin induces differentiation when it translocates into the nucleus and binds TCFs to activate downstream targets (middle). Addition of XAV or IWR-1 stabilizes Axin2. Stabilized Axin2 binds β-catenin and retains it in the cytoplasm, resulting in self-renewal through a yet unknown mechanism (right).

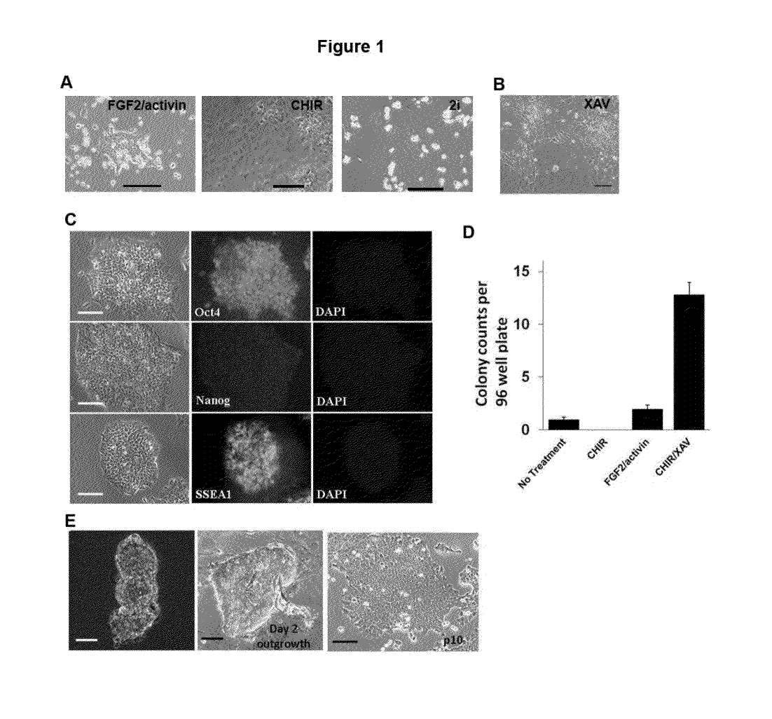

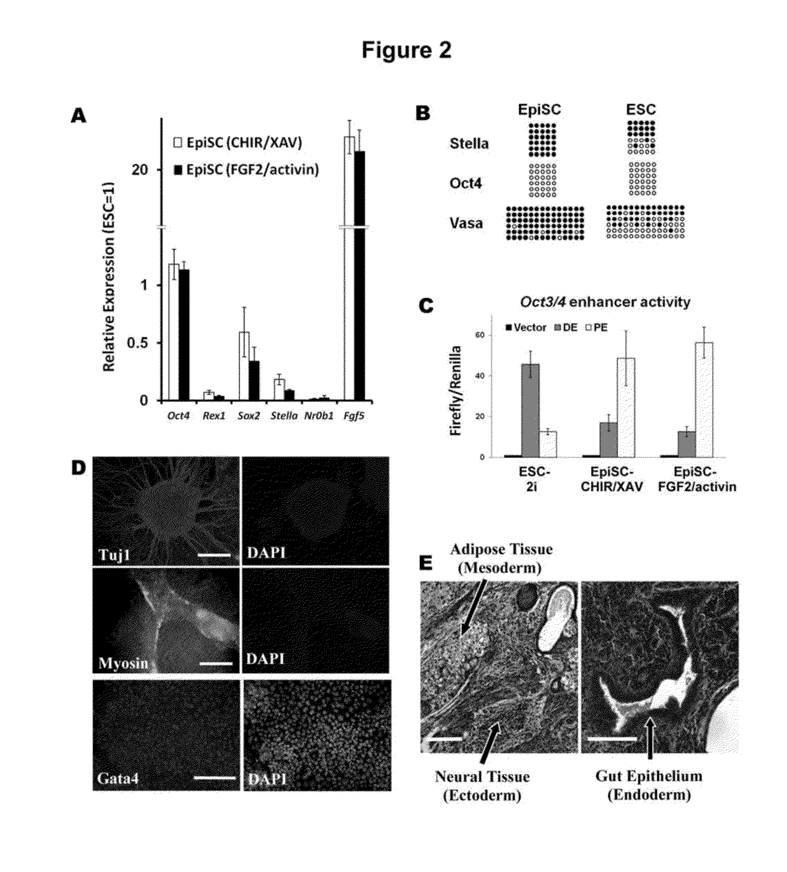

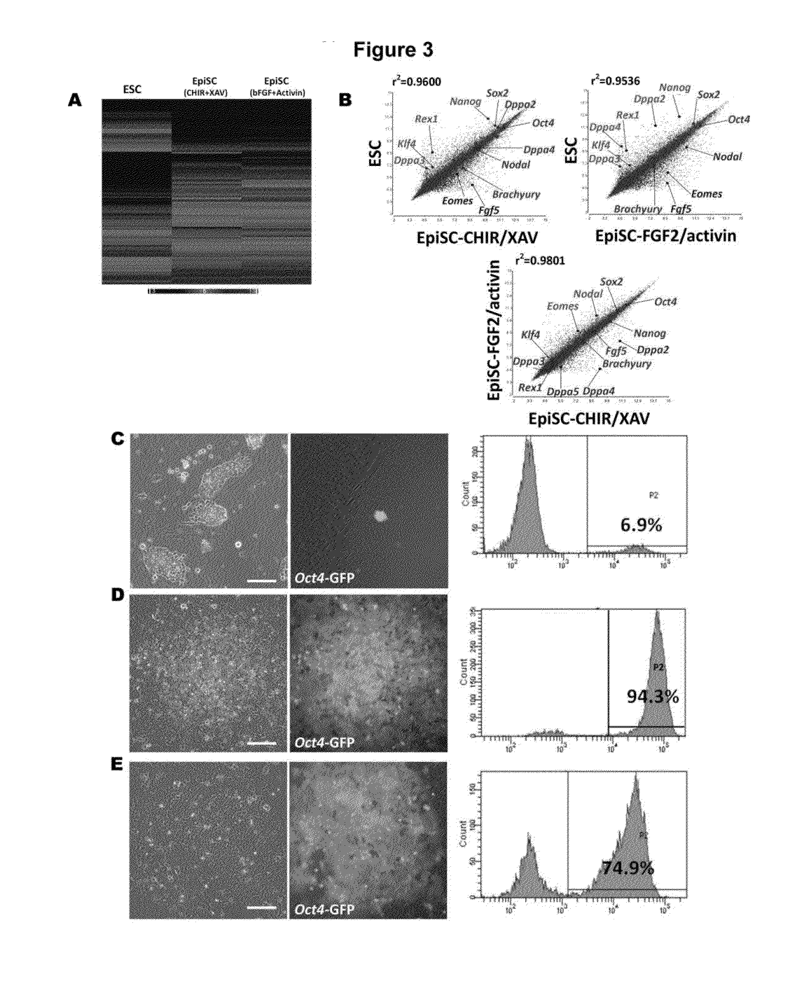

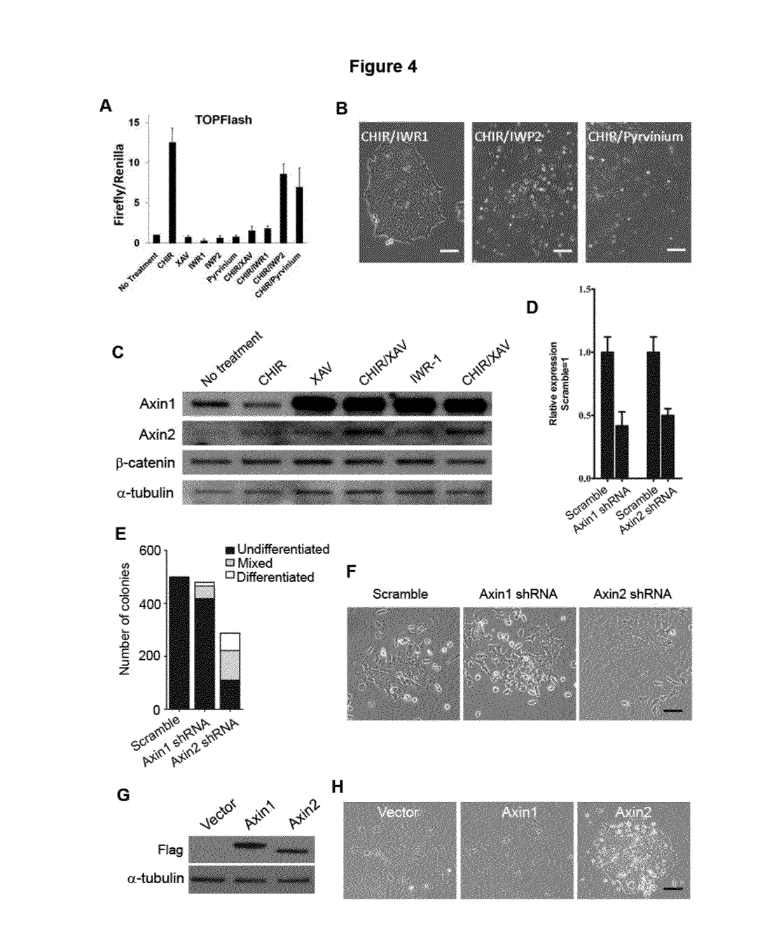

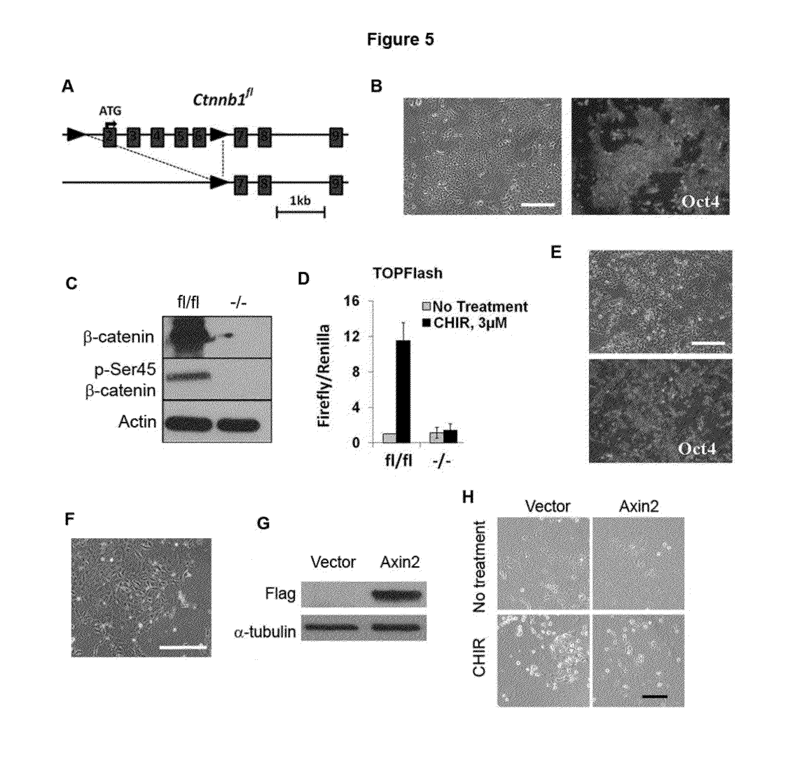

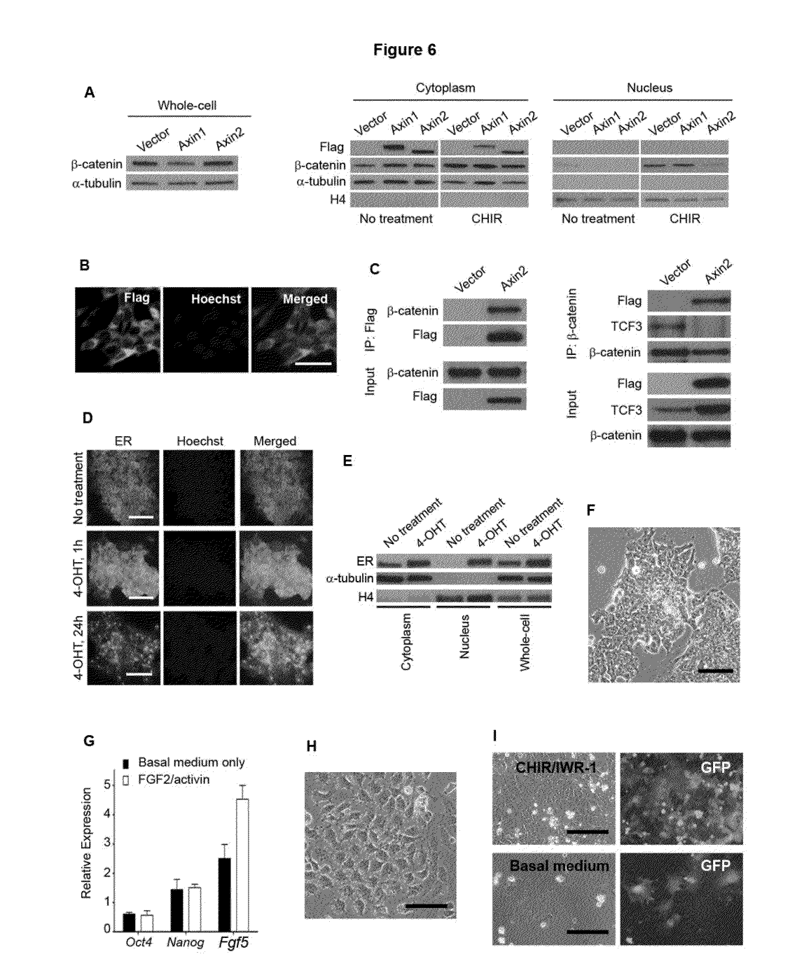

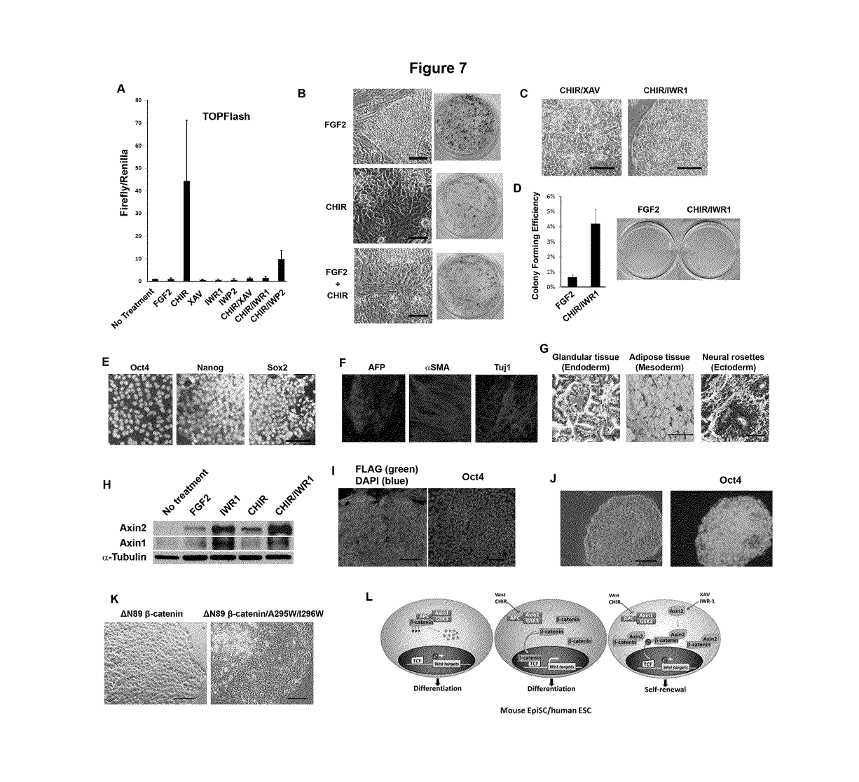

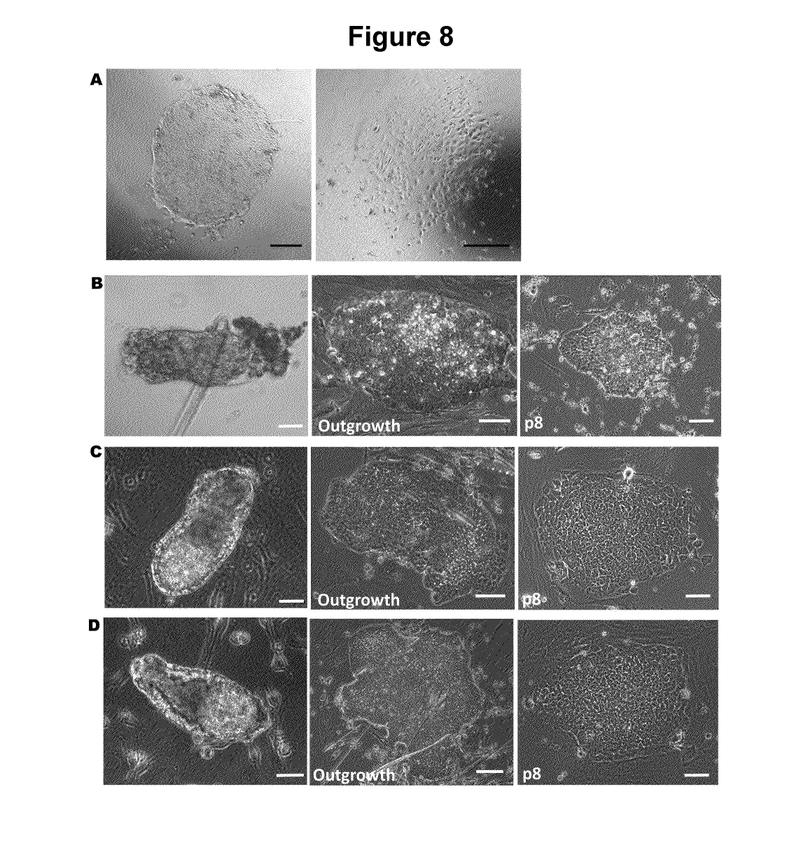

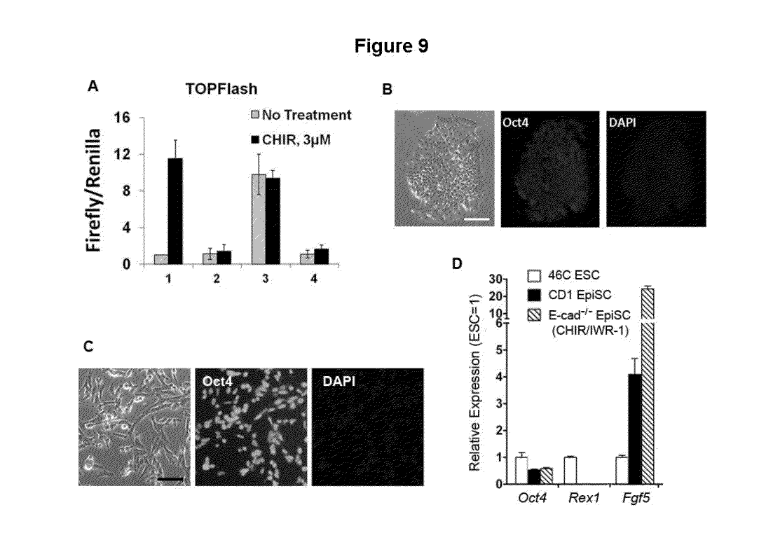

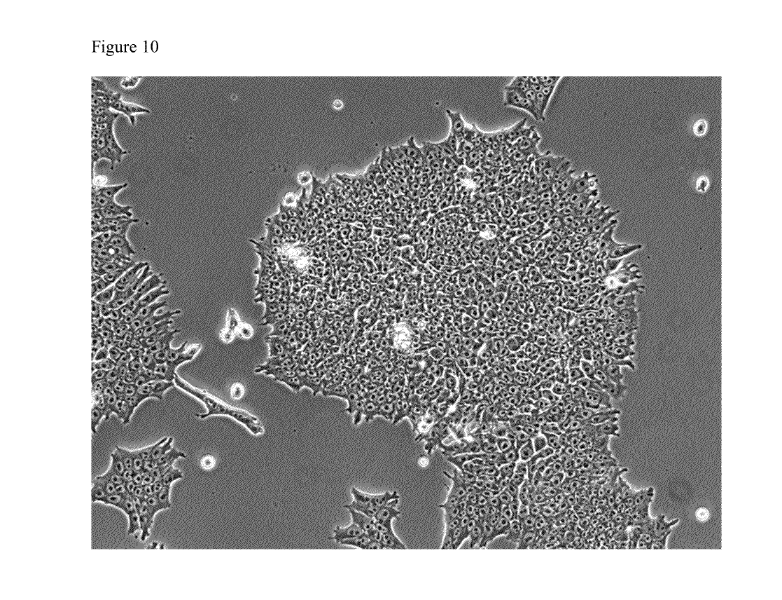

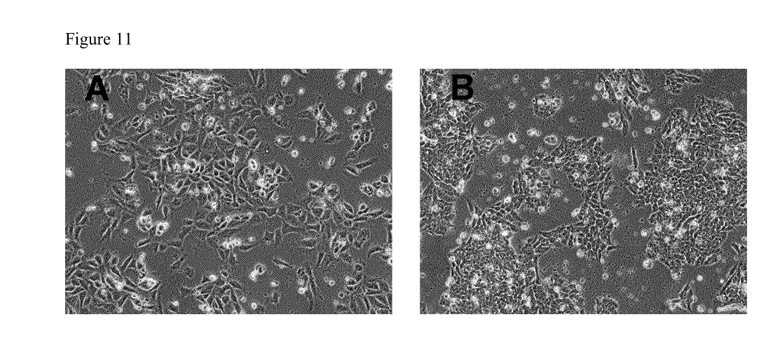

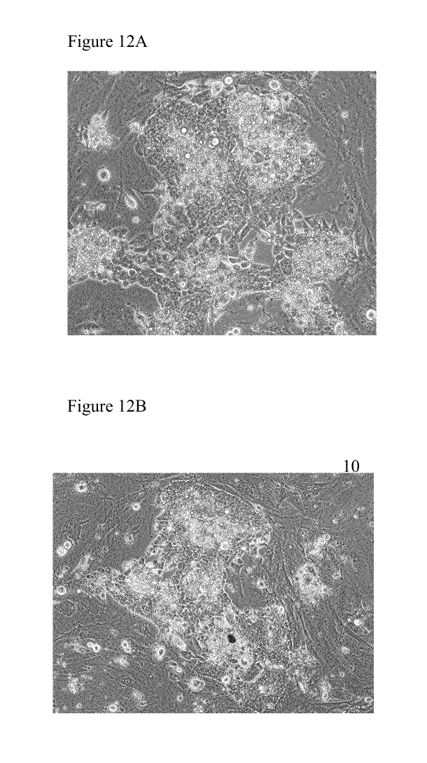

FIG. 8. CHIR/XAV Supports Clonal Growth and De Novo Derivation of EpiSCs, Related to FIG. 1

(A) Left panel: phase-contrast image showing an undifferentiated colony formed from a single CD1 mouse EpiSC deposited into one well of a 96-well plate and cultured in CHIR/XAV. Right panel: phase-contrast image showing a fully differentiated colony formed from a single CD1 mouse EpiSC deposited into one well of a 96-well plate and cultured in CHIR only. No undifferentiated colonies formed under this condition. (B) Epiblast tissue (left panel, outlined in dashed line) of the E5.75 129SvE mouse embryo was dissected and cultured in CHIR/XAV. The outgrowth formed from the plated epiblast (middle panel) was disaggregated to establish a stable EpiSC line (right panel). Three EpiSC lines were established from three plated 129SvE mouse embryos. (C) Derivation of EpiSCs from E7.5 post-implantation Sprague-Dawley rat embryos. Five EpiSC lines were established from seven plated epiblasts. (D) Derivation of EpiSCs from E7.5 Dark Agouti rat embryos. Two EpiSC lines were established from three plated epiblasts. Scale bars, 50 μm (A-D).

FIG. 9. β-Catenin-Mediated EpiSC Self-Renewal Does Not Require Association with TCFs or E-Cadherin, Related to FIG. 6

(A) TOPFlash™ assay in β-catenin^{-/-}+β-catenin mutant EpiSCs. Cells were treated with or without 3 μM CHIR for 12 h. 1, β-catenin^{fl/fl} EpiSCs; 2, β-catenin^{-/-} EpiSCs; 3, β-catenin^{-/-}+ΔNβ-catenin EpiSCs; 4, β-catenin^{-/-}+ΔNβ-catenin/A295W/I296W EpiSCs. Data represent mean±s.d. of three biological replicates. (B) Oct4 immunostaining of β-catenin^{-/-}+ΔNβ-catenin/A295W/I296W EpiSCs maintained in GMEM/10% FBS, passage 21. (C) Oct4 immunostaining of E-cadherin^{-/-} EpiSCs maintained in CHIR/IWR-1, passage 11. (D) qRT-PCR analysis of gene expression in E-cadherin^{-/-} EpiSCs maintained in CHIR/IWR-1 for 5 passages. Data represent mean±s.d. of three biological replicates.

FIG. 10. Mouse EpiSCs Maintained in CHIR99021/53AH.

CD1 mouse EpiSCs were cultured in GMEM/10% FBS medium supplemented with 3 μm CHIR99021 and 1 μM 53AH. The picture shows CD1 EpiSCs after 21 passages in CHIR/53AH.

FIG. 11. Human ESCs Self-Renewal is Maintained in CHIR/53AH.

(A) H9 human ESCs were plated onto Matrigel™-coated dishes and cultured in serum-free N2B27 only. They differentiated after 7 days in culture. (B) H9 human ESCs were plated onto Matrigel™-coated dishes and cultured in serum-free N2B27 supplemented with 3 μM CHIR99021 and 1 μM 53AH. These cells have been maintained in this condition for over 10 passages and still remain undifferentiated.

FIG. 12A-B. Chicken ESC Lines Derived in the CHIR153AR Condition.

The two pictures show two individual ESC-like colonies derived from stage X embryos of Rhode Island Red brown eggs.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments disclosed herein can be combined unless the context clearly dictates otherwise.

As used herein, “about” means+/−5% of the recited parameter.

The present invention relates to compositions, culture media and methods of culturing pluripotent stem cells, such as epiblast stem cells (EpiSCs) and embryonic stem cells (ESCs), in order to culture, derive, and reprogram pluripotent stem cells (such as converting ESCs to EpiSCs). The invention further provides methods for isolating and maintaining homogeneous preparations of pluripotent stem cells.

In a first aspect, the present invention provides compositions, comprising:

(a) a compound that can stabilize axin; and

(b) a compound that can stabilize β-catenin.

In one embodiment, the composition comprises:

(a) an inhibitor of β-catenin binding to T-cell factors (Tcfs); and

(b) a suppressor of glycogen synthase kinase (GSK3) activation.

The compositions of the present invention can be used, for example, as a cell culture media additive that acts synergistically to provide the unexpected benefits of the culture media and methods of the invention. These unexpected benefits are obtained by culturing pluripotent stem cells in the presence of compositions of the invention.

An inhibitor of β-catenin binding to Tcfs can be any compound capable of interfering with such binding. Such inhibition can be partial or complete. As shown in the examples herein, stabilized axin serves to retain β-catenin in the cytoplasm.

Under normal condition, axin is degraded by tankyrase. As used herein, “stabilizing axin” means limiting axin degradation by tankyrase. Such inhibition can be any amount of inhibition, preferably at least a 20% reduction in tankyrase degradation of axin; and preferably at least a 25%, 50%, 75%, 85%, 90%, 95%, 98%, or greater reduction in tankyrase degradation of axin. Similarly, any suitable amount of inhibition of β-catenin binding to T-cell factors can be provided by the methods of the invention, preferably at least 50% inhibition, and more preferably at least 60%, 70%, 80%, 90%, 95%, 98%, or greater inhibition.

Axin protein sequences are provided in SEQ ID NO: 43(human axin 1), SEQ ID NO: 44(human axin 2), SEQ ID NO:45 (mouse axin 1), and SEQ ID NO:46 (mouse axin 2).

The T-cell factor/Lymphocyte enhancer factor-1 (Tcf/Lef-1) family has four members: Tcf1, Tcf3, Tcf4, and Lef1 (human (SEQ ID NOS:1 to 4) and mouse (SEQ ID NOS: 6 to 9, respectively). In a preferred embodiment, the inhibitor inhibits β-catenin binding to all four members of the Tcf/Lef-1. The sequence of the human (SEQ ID NO:5) and mouse (SEQ ID NO:10)β-catenin proteins are also provided.

Exemplary inhibitors of β-catenin binding to Tcfs (and thus which can stabilize axin) include but are not limited to XAV939, IWR-1, 53AH, or salts thereof.

XAV 939 is also known as 3,5,7,8-Tetrahydro-2-[4-(trifluoromethyl)phenyl]-4H-thiopyrano[4,3-d]pyrimidin-4-one (XAV939) and is available, for example, from Sigma Chemical Company. Its structure is as follows:

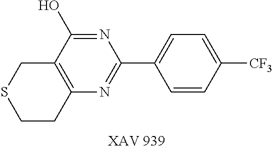 [SMILES: Oc1nc(-c2ccc(C(F)(F)F)cc2)nc2c1CSCC2]

IWR-1 (see below) is also known as 4-(1,3,3a,4,7,7a-Hexahydro-1,3-dioxo-4,7-methano-2H-isoindol-2-yl)-N-8-quinolinyl-Benzamide, and is available, for example, from Sigma Chemical Company.

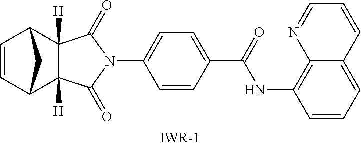

53AH (see below) is also known as (1R,4r)-4-((2s,3aR,4R,7S,7aS)-1,3-dioxooctahydro-1H-4,7-methanoinden-2-yl)-N-(quinolin-8-yl)cyclohexanecarboxamide and is available, for example, from Cellagen Technology. It is an analog of IWR-1, and has been found by the inventors to be particularly effective in promoting mouse EpiSC/human ESC self-renewal when combined with GSK3 inhibitors. The 53AH structure is shown below.

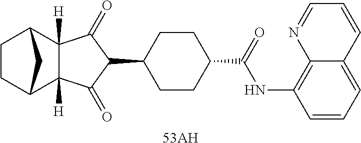 [SMILES: O=C1C([C@H]2CC[C@H](C(=O)Nc3cccc4cccnc34)CC2)C(=O)[C@H]2[C@H]3CC[C@H](C3)[C@@H]12 ; "53AH"]

The inhibitor of β-catenin binding to Tcfs may be present in the composition or cell culture medium in any suitable amount/concentration, depending on the intended use and the specific inhibitor used. In one non-limiting embodiment, XAV939 (or IWR-1 or 53AH) is used at a concentration of between about 1 μM and about 10 μM; in other embodiments, between about 1.5 μM and about 7.5 μM; about 2 μM and about 6 μM; about 2.5 μM and about 5 μM, about 1 μM and about 7.5 μM; about 1 μM and about 5.0 μM; or between about 1 μM and about 2.5 μM; or about 1 μM; or about 2 μM. Thus, XAV939 (or IWR-1 or 53AH) may be present in the compositions in any amount that permits adding to cell culture medium to provide a concentration of between about 1 μM and about 10 μM.

A suppressor of GSK3 activation is any compound capable of inhibiting the kinase activity of one or more members of the GSK3 family (and which thus stabilizes β-catenin). Such inhibition can be partial or complete. As shown in the examples herein, stabilized β-catenin can be retained in the cytoplasm.

Under normal condition, β-catenin is degraded by GSK3. GSK3 is a constitutively active, ubiquitous expressed serine/threonine kinase. GSK-3 can phosphorylate beta-catenin, targeting it for degradation Inhibition of GSK3 therefore can stabilize beta-catenin. As used herein, “stabilizing β-catenin” means limiting β-catenin degradation by GSK3. Such inhibition can be any amount of inhibition, preferably at least a 20% reduction in GSK3 degradation of β-catenin; and preferably at least a 25%, 50%, 75%, 85%, 90%, 95%, 98%, or greater reduction in GSK3 degradation of β-catenin. Similarly, any suitable amount of suppression of GSK activation can be provided by the methods of the invention, preferably at least 20% suppression, and more preferably at least 30%, 40%, 50%, 60%, 70%, 80%, 90%, 95%, 98%, or greater suppression.

The GSK3 enzyme family is known to those of skill in the art and includes, but is not limited to, GSK3-α and GSK3-β (human (SEQ ID NOS 11 to 12) and mouse (SEQ ID NOS 13 to 14)).

Any suitable suppressor of GSK3 activation (non-ATP competitive inhibitors and ATP competitive inhibitors) can be used, including but not limited to CHIR98014, CHIR99021, Wnt3a, AR-AO144-18, TDZD-8, SB216763, and SB415286. In one embodiment, the suppressor of GSK3 activation comprises CHIR99021 (Stemgent), or salts thereof. CHIR99021 is also known as 6-42-44-(2,4-Dichlorophenyl)-5-(4-methyl-1H-imidazol-2-yl)pyrimidin-2-yl)amino)ethyl)amino)nicotinonitrile, and its structure is as follows:

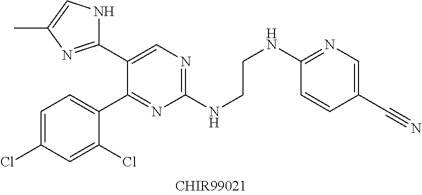 [SMILES: Cc1c[nH]c(-c2cnc(NCCNc3ccc(C#N)cn3)nc2-c2ccc(Cl)cc2Cl)n1]

CHIR 98014 is 2,6-Pyridinediamine, N6-[2-[[4-(2,4-dichlorophenyl)-5-(1H-imidazol-1-yl)-2-pyrimidinyl]amino]ethyl]-3-nitro- (Axon Medchem). TDZD-8 is benzyl-2-methyl-1,2,4-thiadiazolidine-3,5-dione (available from Sigma Chemical Co., St. Louis, Mo.). SB216763 is 3-(2,4-Dichlorophenyl)-4-(1-methyl-1H-indol-3-yl)-1H-pyrrole-2,5-dione (available from Sigma Chemical Co., St. Louis, Mo.). SB415286 is 3-[(3-Chloro-4-hydroxyphenyl)amino]-4-(2-nitrophenyl)-1H-pyrrol-2,5-dione (available from Sigma Chemical Co., St. Louis, Mo.).

Wnt3a is a protein that can be added to the cell culture at any suitable concentration. In one non-limiting embodiment, the Wnt3a is added to the cell culture medium at a concentration of between about 20 ng/ml to about 200 ng/ml. The amino acid sequence of human and mouse Wnt3a is provided in SEQ ID NOS: 15 and 16.

AR-AO144-18 is N-[(4-Methoxyphenyl)methyl]-N′-(5-nitro-2-thiazolyl), and is available from Toronto Research Chemicals, Inc. It can be used in the cell culture at any suitable concentration.

The GSK3 suppressor may be present in the composition or cell culture medium in any suitable amount/concentration, depending on the intended use and the specific GSK3 suppressor used. In one embodiment, CHIR99021 is present in the resulting cell culture medium at a concentration of between about 1 μM and about 10 μM; in other embodiments, between about 1.5 μM and about 7.5 μM; about 2 μM and about 6 μM; and about 2.5 μM and about 5 μM. Thus, CHIR99021 may be present in the compositions in any amount that permits adding to cell culture medium to provide a concentration of between about 1 μM and about 10 μM.

In another embodiment, the inhibitor of β-catenin binding to Tcfs and the suppressor of GSK activation are present in the culture media at a molar ratio of between 1:3 and 3:1.

In one specific embodiment, the inhibitor of β-catenin binding to Tcfs (compound that can stabilize axin) is (1R,4r)-4-((2s,3aR,4R,7S,7aS)-1,3-dioxooctahydro-1H-4,7-methanoinden-2-yl)-N-(quinolin-8-yl)cyclohexanecarboxamide (53AH) or a salt thereof, and the suppressor of GSK3 activation (compound capable that can stabilize (β-catenin) is 6-((2-((4-(2,4-Dichlorophenyl)-5-(4-methyl-1H-imidazol-2-yl)pyrimidin-2-yl)amino)ethyl)amino)nicotinonitrile CHIR99021 or a salt thereof.

In these various embodiments, the compositions or cell culture media of the invention can be provided as a liquid, or may comprise a concentrate that can be reconstituted by an end user, for example, by mixing with basal medium. If provided as a liquid, the cell culture media should be shielded from light.

The culture media of the invention may contain other components as appropriate for a given application, including but not limited to basal medium (ie: any medium that supplies essential sources of carbon and/or vitamins and/or minerals for pluripotent stem cell growth) such as Glasgow minimal essential medium (GMEM), Dulbecco's Modified Eagle Medium (DMEM), and the like. The basal medium is typically free of protein and incapable on its own of supporting self-renewal of ES cells. Other components that may be added include, but are not limited to, a protein source (including but not limited to fetal bovine serum, serum albumin (purified or recombinant), serum replacements, etc.), an iron transporter (provides a source of iron or provides ability to take up iron from the culture medium) such as transferrin or apotransferrin; a carbohydrate source (including but not limited to sodium pyruvate) a source of additional amino acids (such as MEM non-essential amino acids and L-glutamine), and reducing agents (such as 2-mercaptoethanol)

In a further embodiment of any of the above embodiments, the media may further comprise a factor promoting survival and/or metabolism of the cells, including but not limited to insulin or insulin-like growth factors.

In a further embodiment, the culture media of the invention is made as described in the examples that follow.

Unless the context clearly indicates otherwise, all embodiments of this first aspect can be used in combination with each, and can also be used in the various further aspects of the invention discussed below.

In a second aspect, the present invention provides a method of culturing pluripotent stem cells, comprising culturing the cells in the culture medium of any embodiment or combination of embodiments of the first aspect of the invention under conditions suitable to maintain pluripotent stem cell self-renewal.

The methods of the invention permit extended passaging of the pluripotent stem cells. While not being bound by any specific mechanism of action, the inventors believe that stabilized Axin2 retains stabilized β-catenin in the cytoplasm, preventing it from entering the nucleus and binding to T-cell factors therein, and that the stabilization of β-catenin and its retention in the cytoplasm is sufficient to maintain pluripotent stem cell self-renewal. Thus, any method that retains stabilized β-catenin in the cytoplasm should also work for maintaining pluripotent stem cell self-renewal.

In a third aspect, the methods of the invention can be used to derive a pluripotent cell line from a tissue, comprising

(a) culturing a tissue comprising a pluripotent cell in a culture medium of any embodiment or combination of embodiments of the first aspect of the invention; and

(b) isolating the pluripotent cells in a culture medium of any embodiment or combination of embodiments of the first aspect of the invention.

This aspect permits derivation of new stem cell lines from a tissue (including, but not limited to, a blastocyst, fertilized embryo, inner cell mass (ICM), or adult tissue). In one exemplary embodiment, as per standard derivation protocol, mouse blastocysts are plated on gelatinized tissue culture dishes containing the medium. The blastocysts are allowed to attach and grow for several days in incubation. The outgrowths of blastocysts are then disaggregated and expanded until they are verified as a mES cell line. Verification is demonstrated following a minimum of 10 passages by marker analysis for pluripotency, growth characteristics, genetic modification proof of concept, EB formation assays to demonstrate differentiation potential and germline competency assay by blastocyst injection and subsequently mating of resulting chimeras.

Any type of pluripotent stem cell can be used with the methods of the invention, such as EpiSCs and ESCs. Stem cell densities for the methods of the invention vary according to the pluripotent stem cells being used and the natures of any desired progeny. In one embodiment, the stem cells are cultured as described in the examples that follow. The pluripotent stem cell may be from any species, including but not limited to mammals such as mice, rats, cows, rabbits, pigs, humans, and chickens.

Those of skill in the art understand how to identify ES cells by analysis of ES cell markers, including but not limited to expression of alkaline phosphatase, Oct4, Nanog, Rex1, and Sox2, and SSEA1. The methods may also comprise transfecting stem cells with a selectable markers and selecting stem cells with the desired phenotype.

Stem cell densities for the methods of the invention vary according to the pluripotent stem cells being used and the natures of any desired progeny. In one embodiment, the stem cells are cultured in a monolayer on a cell surface.

Any suitable surface of a desired size can be used for culturing stem cells, including but not limited to plastics, metal, and composites. In one embodiment, plastic tissue culture plates are used. In another embodiment, the cell culture surface comprises a cell adhesion protein coated on the culture surface. Any suitable cell adhesion protein can be used, including but not limited to gelatin.

In one specific embodiment, the inhibitor of β-catenin binding to Tcfs (compound that can stabilize axin) is (1R,4r)-4-((2s,3aR,4R,7S,7aS)-1,3-dioxooctahydro-1H-4,7-methanoinden-2-yl)-N-(quinolin-8-yl)cyclohexanecarboxamide (53AH) or a salt thereof, and the suppressor of GSK3 activation (compound capable that can stabilize (β-catenin) is 6-((2-((4-(2,4-Dichlorophenyl)-5-(4-methyl-1H-imidazol-2-yl)pyrimidin-2-yl)amino)ethyl)amino)nicotinonitrile (CHIR99021) or a salt thereof. Exemplary concentrations of the inhibitors that can be used are described herein. In another embodiment, the inhibitor of β-catenin binding to Tcfs and the suppressor of GSK activation are present in the culture media at a molar ratio of between 1:3 and 3:1.

General pluripotent stem cell culture conditions are well known to those of skill in the art. Specific conditions for a given cell culture will depend on all relevant factors, including the cell type, the inhibitors used, the amount of cells desired, and other specifics of the study. Any of the cell culture media disclosed in the first aspect of the invention can be used in this embodiment as well. Exemplary such methods are described in the examples that follow.

In another aspect, the present invention provides stem cells obtained by the methods of any embodiment of the invention. Stem cells of the invention can be used, for example, in assays for drug discovery and for cell therapy.

In another aspect, the present invention provides kits comprising

(a) a first container comprising an inhibitor of (β-catenin binding to Tcf3 or compound that can stabilize axin; and

(b) a second container comprising a suppressor of GSK activation, or compound capable that can stabilize β-catenin.

The kits may comprise any embodiment or combination of embodiments of the compositions of the invention disclosed above. The kits can be used to prepare/reconstitute the culture media of the invention. The kit may further comprise any one or more components of any of the embodiments described above.

### Example 1

### Retention of Stabilized β-Catenin in the Cytoplasm Maintains Mouse Epiblast Stem Cell and Human Embryonic Stem Cell Self-Renewal

### Summary

Wnt/β-catenin signaling plays a variety of roles in regulating stem cell fates. Its specific role in mouse epiblast stem cell (EpiSC) and human embryonic stem cell (ESC) self-renewal, however, remains poorly understood. Here, we show that Wnt/β-catenin functions in both self-renewal and differentiation in mouse EpiSCs and human ESCs. Stabilization and nuclear translocation of (β-catenin and its subsequent binding to T-cell factors (TCFs) induces differentiation. Conversely, stabilization and retention of β-catenin in the cytoplasm maintains self-renewal. Cytoplasmic retention of (β-catenin is affected by stabilization of Axin2, a downstream target of (β-catenin, or by genetic modifications to β-catenin that prevent its nuclear translocation. Our results reveal a novel mechanism by which (β-catenin mediates stem cell self-renewal, one that will have broad implications in understanding the regulation of stem cell fate.

### Introduction

Mouse epiblast stem cells (EpiSCs) are pluripotent stem cells derived from post-implantation epiblasts, and share several properties with mouse embryonic stem cells (ESCs), including the expression of core pluripotency factors Oct4, Nanog and Sox2, and the ability to differentiate into all three primary germ layers even after long-term culture (Brons et al., 2007; Tesar et al., 2007). Despite these similarities, mouse EpiSCs and

ESCs differ significantly in their requirements for self-renewal. Mouse ESC self-renewal is normally mediated by the activation of signal transducer and activator of transcription 3 (STAT3) by leukemia inhibitory factor (LIF) (Niwa et al., 1998), whereas mouse EpiSCs are non-responsive to LIF/STAT3 signaling and instead require the cytokines fibroblast growth factor 2 (FGF2) and activin A for self-renewal (Brons et al., 2007; Tesar et al., 2007). Unlike mouse ESCs, which can be efficiently propagated from dissociated single cells, mouse EpiSCs cultured in FGF2/activin survive poorly upon single cell dissociation, and therefore are routinely passaged as small clumps. The lower viability of dissociated EpiSCs suggests that signaling pathways other than FGF2/activin might be involved in regulating EpiSC self-renewal, and that these pathways are insufficiently activated in the FGF2/activin condition. We considered Wnt/β-catenin as one such candidate pathway.

In the absence of Wnt ligand, β-catenin, the key mediator of the canonical Wnt/13-catenin pathway, is phosphorylated by glycogen synthase kinase 3 (GSK3), leading to proteasome-mediated degradation of β-catenin. When Wnt ligand binds to its receptor complex, composed of Frizzled and low-density-lipoprotein-receptor-related protein 5 or 6, the canonical Wnt/β-catenin pathway is activated, leading to the inhibition of GSK3 and the stabilization of β-catenin. Stabilized β-catenin then translocates to the nucleus, where it interacts with T-cell factors (TCFs) to regulate gene expression. Activation of Wnt/β-catenin signaling produces diverse and sometimes opposite outcomes in different cell types, and it has therefore been proposed that Wnt/β-catenin might regulate cell fates in a context- and cell type-dependent manner (Sokol, 2011). How activation of the same Wnt/β-catenin signal yields disparate effects in different cell types, however, remains poorly understood.

Mouse EpiSCs can be maintained as a homogeneous population and genetically modified without changes to their identity, and therefore provide an ideal model system for determining whether or not Wnt/β-catenin regulates stem cell fates through a context- and stage-dependent manner, and if so, how this might occur. Here, we reveal a novel mechanism by which Wnt/β-catenin regulates stem cell fates. Wnt/β-catenin signaling promotes mouse EpiSC self-renewal when stabilized β-catenin is retained in the cytoplasm, and induces differentiation if β-catenin translocates into the nucleus and binds TCFs. Wnt/β-catenin also regulates human ESC fate through a mechanism similar to that in mouse EpiSCs, supporting the notion that human ESCs are more closely related to mouse EpiSCs than to mouse ESCs.

### Results

### Combined Use of CHIR99021 and XAV939 Maintains EpiSC Self-Renewal

Previously, we showed that two small-molecule inhibitors (2i), CHIR99021 (CHIR) and PD0325901, could efficiently maintain mouse ESC self-renewal independent of LIF/STAT3 signaling (Ying et al., 2008). CHIR stabilizes β-catenin through inhibition of GSK3, and PD0325901 suppresses the mitogen-activated protein kinase (MAPK) pathway.

To ascertain whether this inhibitor-based system is also capable of maintaining self-renewal in EpiSCs, we administered CHIR with or without PD0325901 and found that EpiSCs rapidly differentiated or died in both cases (FIG. 1A). We therefore reasoned that if CHIR induces EpiSC differentiation through stabilization of β-catenin, de-stabilization of β-catenin might promote EpiSC self-renewal. We tested this hypothesis by administering the tankyrase inhibitor XAV939 to mouse EpiSC cultures. XAV939-mediated inhibition of tankyrase stabilizes Axin, leading to the formation of the β-catenin destruction complex, composed of GSK3, Axin and adenomatous polyposis coli (APC) (Huang et al., 2009). Mouse EpiSCs remained undifferentiated for approximately 1 week in the presence of XAV939, but differentiated after passaging (FIG. 1B). Surprisingly, dual administration of CHIR and XAV939 (“CHIR/XAV” hereafter) allowed long-term maintenance of undifferentiated EpiSCs without exogenous growth factors or cytokines (FIG. 1C). EpiSCs cultured in CHIR/XAV could be routinely passaged by single-cell dissociation and replating onto gelatin-coated dishes, and could be cryo-preserved and recovered at high efficiency by standard techniques.

We compared the clonogenicity of EpiSCs cultured in different conditions. Approximately 13% of individual EpiSCs plated onto gelatin-coated 96-well plates and cultured in CHIR/XAV formed morphologically-undifferentiated colonies. This colony formation frequency is approximately six times greater than that of EpiSCs cultured in FGF2/activin (FIGS. 1D and 8A). EpiSC colonies formed in CHIR/XAV were readily expanded to establish stable cell lines. The high propagation efficiency of EpiSCs in CHIR/XAV prompted us to test the derivation of EpiSC lines de novo. As expected, in the CHIR/XAV condition, EpiSCs were readily derived from embryonic day (E) 5.75 embryos of CD1 and 129SvE mice (FIGS. 1E and 8B). EpiSCs were also established from E7.5 Sprague-Dawley and Dark Agouti rat embryos using CHIR/XAV (FIGS. 8C and 8D).

### EpiSCs Derived and Maintained in CHIR/XAV Exhibit the Molecular Hallmarks of EpiSCs

To determine whether the cells derived and maintained in the CHIR/XAV condition retain an EpiSC identity, we examined their molecular signatures and their differentiation potential. These cells expressed Oct4 and Sox2, the key pluripotency genes, and Fgf5, a post-implantation epiblast-specific marker (Brons et al., 2007; Tesar et al., 2007). Their expression of Rex1, Nr0b1, and Stella, markers for the pre-implantation epiblast and primordial germ cells (PGCs), was significantly lower than that of ESCs (FIG. 2A). In EpiSCs maintained in CHIR/XAV, the Oct4 promoter was unmethylated, while promoter regions of Stella and Vasa, specific markers for ESCs and PGCs, were heavily methylated (FIG. 2B). EpiSCs maintained in CHIR/XAV showed strong activity in the Oct4-proximal enhancer, which is preferentially active in EpiSCs, in contrast with ESCs, which mainly exhibit activity in the distal enhancer (Bao et al., 2009; Yeom et al., 1996) (FIG. 2C). EpiSCs readily formed embryoid bodies (EBs) in suspension culture upon withdrawal of CHIR/XAV and differentiated into cell types representative of all three embryonic germ layers (FIG. 2D). We injected CD1 mouse EpiSCs derived and maintained in CHIR/XAV into two SCID mice. Teratomas containing tissues of all three embryonic germ layers were formed in both mice (FIG. 2E). We also tested the chimera formation ability of these CD1 EpiSCs by injecting them into C57BL/6 mouse blastocysts. No chimeras ensued from 58 blastocysts injected, an outcome consistent with previous observations (Brons et al., 2007; Tesar et al., 2007).

To further establish the identity of EpiSCs maintained in CHIR/XAV, we performed whole-genome microarray analyses. EpiSCs derived and grown in CHIR/XAV or FGF2/activin exhibited similar gene expression patterns; these patterns were distinct from those of mouse ESCs (FIG. 3A). Notably, expression of some ESC-specific genes, including Dppa2, Dppa4, and Dppa5a (Han et al., 2010; Maldonado-Saldivia et al., 2007), was up-regulated while expression of the differentiation-associated genes Eomes and Nodal was down-regulated in EpiSCs maintained in CHIR/XAV compared to EpiSCs in FGF2/activin (FIG. 3B). These results suggest that although EpiSCs in CHIR/XAV exhibit key EpiSC features, they might be developmentally closer to ESCs than to EpiSCs grown in FGF2/activin. We took advantage of Oct4-GFP EpiSCs to explore this prospect further. The GFP transgene in Oct4-GFP EpiSCs is under the control of an 18 kb genomic Oct4 segment containing the entire regulatory region of the Oct4 gene (Yeom et al., 1996). Oct4-GFP-positive and -negative EpiSCs represent E5.5 early-stage and E6.5 late-stage in vivo epiblast cells, respectively (Han et al., 2010). We purified Oct4-GFP-positive EpiSCs and cultured them in CHIR/XAV or FGF2/activin. The percentage of Oct4-GFP-positive cells decreased to approximately 5% during 7 passages in FGF2/activin (FIG. 3C). In CHIR/XAV, however, approximately 95% of EpiSCs were still GFP-positive after 7 passages, and approximately 75% were GFP-positive at passage 21 (FIGS. 3D and 3E). These results confirm that EpiSCs representing early-stage in vivo epiblasts are preferentially maintained in CHIR/XAV, whereas EpiSCs representing late-stage in vivo epiblasts are the dominant populations in FGF2/activin.

### CHIR/XAV Promotes EpiSC Self-Renewal Through Stabilization of Axin2

Next, we investigated the mechanism by which CHIR/XAV promotes EpiSC self-renewal. By inhibiting GSK3 phosphorylation of β-catenin, CHIR stabilizes β-catenin, which then trans-locates to the nucleus and forms complexes with DNA-binding proteins, including TCFs, to activate transcription (Logan and Nusse, 2004). As expected, CHIR strongly induced (β-catenin/TCF-responsive TOPFlash™ reporter activity in mouse EpiSCs; the addition of XAV abolished the TOPFlash activity induced by CHIR (FIG. 4A). We tested another small molecule, IWR-1, which, like XAV, also inhibits Wnt/13-catenin signaling through stabilization of Axin (Chen et al., 2009). IWR-1 blocked TOPFlash™ reporter activity induced by CHIR and both inhibitors together promoted EpiSC self-renewal (FIGS. 4A and 4B). In contrast, IWP-2 and Pyrvinium, two small molecules that inhibit Wnt/β-catenin signaling through Axin stabilization-independent mechanisms (Chen et al., 2009; Thorne et al., 2010), were unable to support EpiSC self-renewal (FIGS. 4A and 4B). These results prompted us to examine whether stabilization of Axin is necessary for EpiSC self-renewal promoted by XAV or IWR-1. Axin has two isoforms, Axin1 and Axin2. As expected, XAV or IWR-1 treatment significantly increased the amounts of both Axin1 and Axin2 in mouse EpiSCs (FIG. 4C). The expression level of Axin2, but not Axin1, was also elevated by CHIR treatment (FIG. 4C), an outcome consistent with previous findings that Axin2 is a direct downstream target of Wnt/β-catenin signaling (Jho et al., 2002). As expected, combined use of CHIR with either XAV or IWR-1 further increased the quantity of Axin2 protein in EpiSCs (FIG. 4C). To determine whether Axin mediates EpiSC self-renewal in CHIR/XAV or CHIR/IWR-1, we designed small hairpin RNAs (shRNAs) to knockdown Axin1 and Axin2. Interestingly, knockdown of Axin2, but not Axin1, impaired the self-renewal-promoting effect of CHIR/IWR-1 (FIG. 4D-F). The self-renewal of EpiSCs maintained in FGF2/activin, however, was unaffected by Axin1 or Axin2 knockdown (data not shown). To further confirm the role of Axin, we established mouse EpiSCs overexpressing Axin1 (Axin1-EpiSCs) or Axin2 (Axin2-EpiSCs) in the FGF2/activin condition (FIG. 4G). CHIR alone was sufficient to support robust and long-term expansion of undifferentiated Axin2-EpiSCs following the removal of FGF2/activin. In contrast, Axin1-EpiSCs rapidly differentiated in the presence of CHIR after the removal of FGF2/activin (FIG. 4H). Taken together, these results suggest that Axin2 is the key mediator of EpiSC self-renewal promoted by CHIR/IWR-1 or CHIR/XAV.

### Axin2 Mediates EpiSC Self-Renewal Through Retention of 13-Catenin in the Cytoplasm

Next, we investigated how Axin2 mediates EpiSC self-renewal. First, we sought to determine whether β-catenin is required for EpiSC self-renewal mediated by Axin2. We derived EpiSCs from mouse embryos carrying floxed alleles for β-catenin (FIGS. 5A and 5B). Stable β-catenin^{-/-} EpiSC lines were generated from these β-catenin^{fl/fl} EpiSCs by transient transfection of Cre recombinase, and could be routinely maintained in FGF2/activin. Loss of β-catenin in these β-catenin^{-/-} EpiSCs was confirmed by Western blot analysis and the TOPFlash™ reporter assay (FIGS. 5C and 5D). β-catenin^{-/-} EpiSCs remained undifferentiated even after long-term culture in FGF2/activin (FIG. 5E). However, they differentiated after the removal of FGF2/activin even in the presence of CHIR/XAV or CHIR/IWR-1 (FIG. 5F), suggesting that EpiSC self-renewal in CHIR/XAV or CHIR/IWR-1 is likely mediated by β-catenin. To further confirm the role of β-catenin, we generated β-catenin^{-/-} EpiSCs overexpressing Axin2 in the FGF2/activin condition (FIG. 5G). These cells differentiated after the removal of FGF2/activin, even in the presence of CHIR (FIG. 5H), suggesting that EpiSC self-renewal maintained by Axin2 is also β-catenin-dependent.

Next, we investigated how β-catenin mediates EpiSC self-renewal. Nuclear translocation of β-catenin and its subsequent binding to TCFs have been considered essential events in canonical Wnt/β-catenin signaling. To determine whether nuclear translocation of β-catenin is affected by Axin, we analyzed β-catenin protein levels in whole-cell, cytoplasmic and nuclear fractions before and after CHIR treatment. The amounts of total and cytoplasmic β-catenin protein in Axin2-EpiSCs were comparable to those in EpiSCs transfected with vector only (vector-EpiSCs) (FIG. 6A); however, nuclear β-catenin in Axin2-EpiSCs was barely detectable before or after CHIR treatment, while CHIR treatment dramatically increased the nuclear β-catenin protein level in vector-EpiSCs and Axin1-EpiSCs (FIG. 6A). These results suggest that Axin2 overexpression does not lead to β-catenin degradation, but instead blocks nuclear translocation of β-catenin induced by CHIR. In Axin2-EpiSCs, Axin2 expression was mainly detected in the cytoplasm (FIG. 6B). Axin2 and β-catenin associated with each other, as shown by co-immunoprecipitation (Co-IP) (FIG. 6C); however, the binding between β-catenin and TCF3 was barely detectable in Axin2-EpiSCs, even in the presence of CHIR (FIG. 6C). Taken together, these results suggest that Axin2 binds β-catenin and retains it in the cytoplasm, preventing its nuclear translocation and binding to TCFs.

To determine whether retention of β-catenin in the cytoplasm is necessary and sufficient for EpiSC self-renewal mediated by Axin2, we introduced a floxed ΔNβ-catenin-ERT2 transgene into mouse EpiSCs. ΔNβ-catenin-ERT2 is a fusion protein containing an N-terminally truncated, stabilized β-catenin and a mutant estrogen ligand-binding domain (ERT2) (Lo Celso et al., 2004). ΔNβ-catenin-ERT2 remains in the cytoplasm, and translocates into the nucleus only when 4-hydroxytamoxifen (4-OHT) is added, as confirmed by immunocytochemistry staining and immunoblotting (FIGS. 6D and 6E). EpiSCs overexpressing ΔNβ-catenin-ERT2 were expanded continuously for more than 25 passages in basal medium without addition of exogenous cytokines or small molecules while retaining an EpiSC identity (FIGS. 6F and 6G). The addition of 4-OHT resulted in rapid differentiation, even in the presence of IWR-1 (FIG. 6H). These results are likely attributable to the presence of the ΔNβ-catenin-ERT2 transgene, since its excision by Cre recombinase was associated with reversion to a wild-type EpiSC-like phenotype (FIG. 6I). Collectively, these results suggest that retention of stabilized β-catenin in the cytoplasm is necessary and sufficient for EpiSC self-renewal mediated by Axin2, and that nuclear β-catenin induces EpiSC differentiation.

To further elaborate the role of β-catenin in EpiSCs, we generated a ΔNβ-catenin mutant containing two point mutations, at A295 and 1296 (referred to as A295W/I296W hereafter). These point mutations render β-catenin unable to bind TCFs as well as Axin and APC (Graham 2000). β-catenin^{-/-} EpiSCs overexpressing ΔNβ-catenin or ΔNβ-catenin/A295W/I296W were established in FGF2/activin. A TOPFlash assay confirmed that ΔNβ-catenin is constitutively active whereas ΔNβ-catenin/A295W/I296W exhibits no TOPFlash activity even in the presence of CHIR (FIG. 9A). β-catenin^{-/-} EpiSCs overexpressing ΔNβ-catenin rapidly differentiated after the removal of FGF2/activin. In contrast, β-catenin^{-/-} EpiSCs overexpressing ΔNβ-catenin/A295W/I296W could be continuously expanded in basal medium without overt differentiation (FIG. 9B). Since nuclear β-catenin is mainly associated with TCFs, our results suggest that EpiSC differentiation induced by nuclear β-catenin is likely mediated by β-catenin-TCF binding; nonetheless, the interaction of β-catenin with Axin and APC conceivably might also contribute to the observed effects.

Next, we investigated whether membrane-bound β-catenin also plays a role in the maintenance of EpiSCs. β-catenin is recruited to the cell membrane mainly through binding to E-cadherin (Orsulic et al., 1999). We converted E-cadherin^{-/-} mouse ESCs to EpiSCs under the FGF2/activin condition. These E-cadherin^{-/-} EpiSCs could be expanded in CHIR/IWR-1, and retained an EpiSC identity (FIGS. 9C and 9D), indicating that membrane-bound β-catenin is likely not required for EpiSC self-renewal mediated by Axin2 and β-catenin.

### Modulating β-Catenin Function Maintains Human ESC Self-Renewal

As human ESCs share defining features with mouse EpiSCs (Hanna et al., 2010; Rossant, 2008; Tesar et al., 2007), we tested whether modulating β-catenin function can also promote human ESC self-renewal. As was the case in mouse EpiSCs, TOPFlash™ reporter activity in H9 human ESCs was strongly induced by CHIR; addition of either XAV or IWR-1 abolished this TOPFlash™ activity while IWP-2 only partially suppressed such activity (FIG. 7A). Next, we examined the effect of CHIR/XAV and CHIR/IWR-1 on human ESC self-renewal. CHIR induced differentiation of H9 human ESCs, even in the presence of FGF2 (FIG. 7B). In contrast, co-administration of CHIR with either XAV or IWR-1 resulted in robust self-renewal of H9 human ESCs (FIG. 7C). We found that CHIR/IWR-1 is more effective than CHIR/XAV in promoting human ESC self-renewal, especially in feeder- and FGF2-free conditions; therefore, we focused on CHIR/IWR-1 for our human ESC study. Supplementation of conventional human ESC medium with CHIR/IWR-1 allowed robust propagation of H9 Human ESCs; moreover, the clonogenicity of H9 human ESCs cultured in CHIR/IWR-1 was significantly greater than that in the FGF2 condition (FIG. 7D). Similar results were obtained in H1 and HES3 human ESCs (data not shown). Human ESCs maintained in the conventional FGF2 condition are often morphologically heterogeneous with occasional spontaneous differentiation, whereas human ESCs in the CHIR/IWR-1 condition were observed to be more homogeneous and exhibited almost no spontaneous differentiation. Moreover, human ESCs maintained in CHIR/IWR-1 express pluripotency markers Oct4, Nanog and Sox2 (FIG. 7E), and retain the ability to differentiate into cells of all three germ layers, both in vitro and in vivo (FIGS. 7F and 7G). These results indicate that CHIR/IWR-1 mediates similar self-renewal responses in human ESCs and mouse EpiSCs.

Next, we investigated whether CHIR/IWR-1 maintains human ESC self-renewal through a mechanism similar to that in mouse EpiSCs. IWR-1 treatment significantly increased the amounts of both Axin1 and Axin2 in human ESCs (FIG. 7H). CHIR induced the expression of Axin2, but not Axin1, and combined use of CHIR with IWR-1 further increased Axin2 protein level (FIG. 7H), an outcome similar to what we observed in mouse EpiSCs. To confirm whether Axin2 also mediates human ESC self-renewal, we stably introduced an Axin2 transgene into H9 human ESCs (Axin2-hESC). As expected, CHIR administrated alone could support stable and long-term self-renewal of Axin2-hESCs (FIG. 7I).

Finally, to determine whether cytoplasmic β-catenin can also mediates human ESC self-renewal, we introduced different β-catenin mutants into HES2 human ESCs. As expected, human ESCs overexpressing ΔNβ-catenin-ERT2 or ΔNβ-catenin/A295W/I296W could be continually passaged without overt differentiation, whereas overexpression of ΔNβ-catenin induced rapid differentiation of human ESCs (FIGS. 7J and 7K). These results suggest that human ESC self-renewal and mouse EpiSC self-renewal are supported by a similar mechanism: increasing cytoplasmic β-catenin level and preventing β-catenin interaction with TCFs.

### Discussion

Our study demonstrates that Wnt/β-catenin signaling can promote self-renewal or differentiation of mouse EpiSCs and human ESCs. The stabilization of β-catenin and its retention in the cytoplasm maintains mouse EpiSC and human ESC self-renewal, whereas nuclear translocation of β-catenin and its subsequent binding to TCFs induces differentiation (FIG. 7L). Our finding that cytoplasmic and nuclear β-catenin pools are both involved in regulating cell fates might provide a rational explanation for some of the diverse and sometimes opposite effects of Wnt/β-catenin observed in different contexts. More importantly, our study reveals a new functional avenue of the canonical Wnt/13-catenin pathway, which current dogma depicts as being functionally defined by nuclear translocation of β-catenin and its subsequent binding to TCFs.

The gene regulatory effects of Wnt/β-catenin pathway are initiated upon binding of fβ-catenin to TCFs in the nucleus. So how is cytoplasmic fβ-catenin involved in regulating cell fates? One possible model of regulation is suggested by the interaction of cytoplasmic β-catenin with cadherin, a-catenin, and actin filaments (Yamada et al., 2005), as these interactions have been shown to play multiple and important roles in regulating cellular organization, cell adhesion, and signal transduction from cell surface to the nucleus. Another possibility is that cytoplasmic β-catenin might hold negative regulators of self-renewal in the cytoplasm, thereby preventing them from entering the nucleus and activating or suppressing transcription of their target genes. In this scenario, cytoplasmic β-catenin would promote stem cell self-renewal by alleviating the self-renewal suppression effect of these negative regulators. This might be the case in mouse EpiSCs and human ESCs in which the persistence of β-catenin in the cytoplasm is associated with self-renewal.

Whether β-catenin binds to Axin and APC is likely unimportant for the self-renewal-promoting effect of β-catenin for mouse EpiSCs and human ESCs. Although cytoplasmic β-catenin-ERT2 can bind to Axin and APC while β-catenin/A295W/I296W cannot, both mutants are able to promote self-renewal. Binding between β-catenin and TCFs, on the other hand, might play a dominant-negative role in mouse EpiSC and human ESC self-renewal. Forced expression of stabilized β-catenin or nuclear translocation of β-catenin-ERT2 induced by 4-OHT induces differentiation. This differentiative effect fails to be realized if β-catenin-TCF binding is precluded by either preventing the entry of β-catenin into the nucleus (ΔNβ-catenin-ERT2) or abolishing the ability of β-catenin to bind TCFs (ΔNβ-catenin/A295W/I296W).

Interestingly, β-catenin-TCF binding is essential for β-catenin-mediated mouse ESC self-renewal (Wray et al., 2011; Ying et al., 2008). Blocking β-catenin-TCF binding has the effect of converting mouse ESCs to EpiSCs (unpublished results). Understanding why β-catenin-TCF binding plays opposite roles in mouse ESC and EpiSC self-renewal will likely provide insights into the mechanism underlying the disparate effects of Wnt/13-catenin signaling in different cell types.

Previous efforts to investigate the exact roles of Wnt/β-catenin signaling in various tissue-specific stem cells and the mechanisms underlying those roles have been hampered by the lack of well-established methods for the maintenance of pure tissue-specific stem cells. In contrast, homogeneous mouse EpiSCs and ESCs can be readily derived and genetically modified without changes to their identity. While these two types of stem cells are closely related developmentally, they are molecularly and functionally different, and therefore provide an ideal model system for determining whether and how Wnt/β-catenin regulates stem cell fates through a context- and stage-dependent manner.

The role of β-catenin in human ESC self-renewal has been controversial. It has been suggested that activation of β-catenin by Wnt ligands or GSK3 inhibitors can promote human ESC self-renewal (Cai et al., 2007; Sato et al., 2004). Other studies showed that Wnt/β-catenin signaling is dispensable for human ESC self-renewal, and that its activation predominantly induces differentiation (Davidson et al., 2012; Dravid et al., 2005). Our finding that activation of β-catenin can promote human ESC self-renewal or differentiation, and that the respective outcome is dictated by whether β-catenin translocates into the nucleus, provides a rational explanation for earlier, seemingly paradoxical results. We found that Knockout™ Serum Replace (KSR), bovine serum albumin, and feeders can all partially block β-catenin-TCF transcriptional activity induced by CHIR (data not shown), presumably by promoting the retention of stabilized β-catenin in the cytoplasm. These were included in the culture conditions in which activation of β-catenin was shown to promote human ESC self-renewal. Some of the contradicted results on the role of β-catenin in human ESC self-renewal, therefore, might be attributable to variations in the subcellular localization of β-catenin under different culture conditions.

Human ESCs self-renewal mediated by FGF2 requires activation of both the PI3K and MAPK pathways (Singh et al., 2012). In the CHIR/IWR-1 culture condition, however, human ESCs remain undifferentiated even in the presence of both PI3K and MAPK inhibitors (data not shown), suggesting that human ESC self-renewal mediated by CHIR/IWR-1 is independent of the PI3K and MAPK pathways. Nevertheless, FGF2 and CHIR/IWR-1 act synergistically to promote human ESC self-renewal. This is noteworthy because in mouse ESCs, LIF and CHIR/PD can also independently promote self-renewal, yet there is a synergistic effect when the two are combined (Ogawa et al., 2006; Wray et al., 2010). Understanding how these different pathways work independently or synergistically to maintain stem cell self-renewal will advance our efforts to better control stem cell fate, which is critical to the future of regenerative medicine.

### Experimental Procedures

### Small-Molecule Inhibitors and Cytokines

The following small-molecule inhibitors and cytokines were used at the indicated

final concentrations: CHIR99021 (3 μM), PD0325901 (1 μM), XAV939 (Sigma, 2 μM), IWR-1 (Sigma, 2.5 μM), IWP-2 (Stemgent, 2.5 μM), Pyrvinium (Sigma, 100 nM), recombinant human FGF2 (PeproTech, 10 ng/ml), and recombinant human activin A (PeproTech, 10 ng/ml). CHIR99021 and PD0325901 were synthesized in the Division of Signal Transduction Therapy, University of Dundee, UK.

### Culture Media for Mouse and Rat EpiSCs, and Human ESCs

The basal medium for mouse EpiSC culture is the conventional mouse ESC medium, which consists of GMEM (Sigma) supplemented with 10% fetal bovine serum (FBS) (Hyclone), 2 mM L-glutamine (Invitrogen), 1 mM sodium pyruvate (Invitrogen), 1% nonessential amino acids (Invitrogen), and 0.1 mM (3-mercaptoethanol. Mouse EpiSCs were derived and maintained in the basal medium supplemented with FGF2/activin, CHIR/XAV, or CHIR/IWR-1. The basal medium for rat EpiSC culture is N2B27 (Tong et al., 2011), which was prepared by mixing 500 ml of DMEM/F12 (Invitrogen) with 500 ml of Neurobasal™ medium (Invitrogen), and adding 5 ml of N2 (Invitrogen), 10 ml of B27 (Invitrogen), 5 ml of Glutamax™ (Invitrogen), and 1 ml of 0.1 M β-mercaptoethanol (Sigma). The basal medium for human ESC culture consists of Knockout™ DMEM/F12 supplemented with 20% KSR (Invitrogen), 1% nonessential amino acids, 2 mM L-glutamine, and 0.1 mM (3-mercaptoethanol. Human ESCs were cultured in the basal medium supplemented with FGF2, CHIR/XAV, or CHIR/IWR-1.

### Derivation and Propagation of EpiSCs

Post-implantation epiblasts were isolated from mouse or rat embryos and dissociated into small clumps as previously described (Chenoweth and Tesar, 2010). Epiblast fragments were placed into 4-well plates pre-coated with 0.1% gelatin (for mouse epiblasts) or pre-seeded with γ-irradiated mouse embryonic fibroblasts (MEFs) (for rat epiblasts) and cultured in either the FGF2/activin or the CHIR/XAV conditions. Emerging EpiSCs were trypsinized and expanded every 2-3 days at a subculture ratio of 1:4. Animal experiments were performed according to the investigator's protocols approved by the University of Southern California Institutional Animal Care and Use Committee.

### Western Blot and Co-IP

Western blotting was performed according to a standard protocol. Nuclear and cytoplasmic proteins were extracted using NE-PER Nuclear protein Extraction Kit (Thermo). For Co-IP, cell extracts were prepared using Nonidet P-40 lysis buffer (50 mM Tris-HCl, pH 7.5, 150 mM NaCl, 0.5% Nonidet P-40, 1 mM EDTA, 10% glycerol, 1 mM Na3VO4, 50 mM NaF, and protease inhibitors). The supernatant was collected and incubated with either anti-β-catenin or anti-Flag antibody for 2 h at 4° C. following incubation with protein A/G Plus™-Agarose (Santa Cruz) for 1 h. The beads were then washed five times with lysis buffer and resuspended in SDS sample buffer. Primary antibodies used include the following: β-catenin (BD Bioscience, 1:2,000), phospho-Ser45 β-catenin (9564, Cell Signaling, 1:500), Axin1 (AF3287, R&D, 1:1,000), Axin 2 (M-20, Santa Cruz, 1:200), histone H4 (2592, Cell Signaling, 1:1,000), ERα (MC-20, Santa Cruz, 1:1,000), TCF3 (M-20, Santa Cruz, 1:1,000), actin (C-11, Santa Cruz, 1:1,000), Flag (F3165, Sigma, 1:2,000), α-tubulin (B-5-1-2, Invitrogen, 1:2,000).

### Immunostaining and AP Staining

Immunostaining was performed according to a standard protocol. Primary antibodies used include the following: Oct4 (C-10, Santa Cruz, 1:200), Sox2 (Y-17, Santa Cruz, 1:200), SSEA-1 (480, Santa Cruz, 1:200), GATA-4 (G-4, Santa Cruz, 1:200), Nanog (R&D Systems, 1:200), βIII-tubulin (Invitrogen, 1:2,000), Myosin (MF-20, DSHB, 1:5), AFP (mouse monoclonal, Sigma), and αSMA (mouse monoclonal, Dako). Alexa™ Flour fluorescent secondary antibodies (Invitrogen) were used at a 1:2,000 dilution. Nuclei were visualized with DAPI or Hoechst. AP staining was performed with an alkaline phosphatase kit (Sigma) according to the manufacturer's instructions.

### Promoter/Enhancer Reporter Assay

For quantifying relative Oct3/4 enhancer activities, pGL3-Oct4 DE and pGL3-Oct4 PE plasmids (gifts from Hans Schöler's lab) were co-transfected with the Renilla vector, using the Amaxa™ Transfection Kit (Lonza). Dual Luciferase Assay (Promega) was performed the following day according to the manufacturer's instructions. For quantifying relative β-catenin/Tcf transcriptional activity, pGL2-SuperTOP™ plasmid (gift from Randall Moon) was co-transfected with the Renilla vector and assayed accordingly.

### Flow Cytometry

Cells were collected by trypsinization, resuspended in N2B27 medium, and filtered through a 40-μm cell strainer (BD Bioscience). GFP-positive cells were analyzed on a FACSAria™/LSR II flow cytometer (BD). Purification of Oct4-GFP-positive EpiSCs was carried out by fluorescence-activated cell sorting (FACS) on a FACSAria™ II cell sorter (BD Bioscience).

### Bisulfite Sequencing

Genomic DNA was extracted with the QIAamp™ DNA Mini Kit (Qiagen). Approximately 500 ng DNA from each sample was treated with the EZ DNA methylation kit (ZYMO) to convert the unmethylated C's to U's. The promoter regions of Oct4, Stella, and Vasa were amplified with primer sets, as previously described (Han et al., 2010), using the Expand™ High-fidelity PCR system (Roche), cloned into the pCR-BluntII-TOPO vector (Invitrogen) and sequenced with the T7-promoter primer.

### DNA Microarray Analysis

Total RNA was extracted with the RNeasy™ Mini Kit (Qiagen). RNA was amplified, labeled, and hybridized to the GeneChip™ Mouse Gene 1.0 ST Array according to standard Affymetrix protocols. A DNA microarray was performed at the University of California, Los Angeles DNA Microarray core facility. The data analysis was performed using Partek Microarray Software.

### Accession Numbers

Microarray data reported in this paper have been deposited in the Gene Expression Omnibus database with the accession number of GSE31461.

### REFERENCES

Bao, S., Tang, F., Li, X., Hayashi, K., Gillich, A., Lao, K., and Surani, M. A. (2009). Epigenetic reversion of post-implantation epiblast to pluripotent embryonic stem cells. Nature 461, 1292-1295.
Brons, I. G., Smithers, L. E., Trotter, M. W., Rugg-Gunn, P., Sun, B., Chuva de Sousa Lopes, S. M., Howlett, S. K., Clarkson, A., Ahrlund-Richter, L., Pedersen, R. A., et al. (2007). Derivation of pluripotent epiblast stem cells from mammalian embryos. Nature 448, 191-195.
Cai, L., Ye, Z., Zhou, B. Y., Mali, P., Zhou, C., and Cheng, L. (2007). Promoting human embryonic stem cell renewal or differentiation by modulating Wnt signal and culture conditions. Cell research 17, 62-72.
Chen, B., Dodge, M. E., Tang, W., Lu, J., Ma, Z., Fan, C. W., Wei, S., Hao, W., Kilgore, J., Williams, N. S., et al. (2009). Small molecule-mediated disruption of Wnt-dependent signaling in tissue regeneration and cancer. Nature chemical biology 5, 100-107.
Chenoweth, J. G., and Tesar, P. J. (2010). Isolation and maintenance of mouse epiblast stem cells. Methods in molecular biology 636, 25-44.
Clevers, H. (2006). Wnt/beta-catenin signaling in development and disease. Cell 127, 469-480.
Davidson, K. C., Adams, A. M., Goodson, J. M., McDonald, C. E., Potter, J. C., Berndt, J. D., Biechele, T. L., Taylor, R. J., and Moon, R. T. (2012). Wnt/beta-catenin signaling promotes differentiation, not self-renewal, of human embryonic stem cells and is repressed by Oct4. Proceedings of the National Academy of Sciences of the United States of America 109, 4485-4490.
Dravid, G., Ye, Z., Hammond, H., Chen, G., Pyle, A., Donovan, P., Yu, X., and Cheng, L. (2005). Defining the role of Wnt/beta-catenin signaling in the survival, proliferation, and self-renewal of human embryonic stem cells. Stem cells 23, 1489-1501.
Han, D. W., Tapia, N., Joo, J. Y., Greber, B., Arauzo-Bravo, M. J., Bernemann, C., Ko, K., Wu, G., Stehling, M., Do, J. T., et al. (2010). Epiblast stem cell subpopulations represent mouse embryos of distinct pregastrulation stages. Cell 143, 617-627.
Hanna, J., Cheng, A. W., Saha, K., Kim, J., Lengner, C. J., Soldner, F., Cassady, J. P., Muffat, J., Carey, B. W., and Jaenisch, R. (2010). Human embryonic stem cells with biological and epigenetic characteristics similar to those of mouse ESCs. Proc Natl Acad Sci USA 107, 9222-9227.
Hasegawa, K., Yasuda, S. Y., Teo, J. L., Nguyen, C., McMillan, M., Hsieh, C. L., Suemori, H., Nakatsuji, N., Yamamoto, M., Miyabayashi, T., et al. (2012). Wnt Signaling Orchestration with a Small Molecule DYRK Inhibitor Provides Long-Term Xeno-Free Human Pluripotent Cell Expansion. Stem Cells Transl Med 1, 18-28.
Huang, S. M., Mishina, Y. M., Liu, S., Cheung, A., Stegmeier, F., Michaud, G. A., Charlat, O., Wiellette, E., Zhang, Y., Wiessner, S., et al. (2009). Tankyrase inhibition stabilizes axin and antagonizes Wnt signalling. Nature 461, 614-620.
Jho, E. H., Zhang, T., Domon, C., Joo, C. K., Freund, J. N., and Costantini, F. (2002). Wnt/beta-catenin/Tcf signaling induces the transcription of Axin2, a negative regulator of the signaling pathway. Molecular and cellular biology 22, 1172-1183.
Lo Celso, C., Prowse, D. M., and Watt, F. M. (2004). Transient activation of beta-catenin signalling in adult mouse epidermis is sufficient to induce new hair follicles but continuous activation is required to maintain hair follicle tumours. Development 131, 1787-1799.
Logan, C. Y., and Nusse, R. (2004). The Wnt signaling pathway in development and disease. Annual review of cell and developmental biology 20, 781-810.
Maldonado-Saldivia, J., van den Bergen, J., Krouskos, M., Gilchrist, M., Lee, C., Li, R., Sinclair, A. H., Surani, M. A., and Western, P. S. (2007). Dppa2 and Dppa4 are closely linked SAP motif genes restricted to pluripotent cells and the germ line. Stem cells 25, 19-28
Niwa, H., Burdon, T., Chambers, I., and Smith, A. (1998). Self-renewal of pluripotent embryonic stem cells is mediated via activation of STAT3. Genes & development 12, 2048-2060.
Nusse, R. (2008). Wnt signaling and stem cell control. Cell research 18, 523-527.
Ogawa, K., Nishinakamura, R., Iwamatsu, Y., Shimosato, D., and Niwa, H. (2006). Synergistic action of Wnt and LIF in maintaining pluripotency of mouse ES cells. Biochemical and biophysical research communications 343, 159-166.
Orsulic, S., Huber, O., Aberle, H., Arnold, S., and Kemler, R. (1999). E-cadherin binding prevents beta-catenin nuclear localization and beta-catenin/LEF-1-mediated transactivation. Journal of cell science 112 (Pt 8), 1237-1245.
Rossant, J. (2008). Stem cells and early lineage development. Cell 132, 527-531.
Sato, N., Meijer, L., Skaltsounis, L., Greengard, P., and Brivanlou, A. H. (2004). Maintenance of pluripotency in human and mouse embryonic stem cells through activation of Wnt signaling by a pharmacological GSK-3-specific inhibitor. Nature medicine 10, 55-63.
Singh, A. M., Reynolds, D., Cliff, T., Ohtsuka, S., Mattheyses, A. L., Sun, Y., Menendez, L., Kulik, M., and Dalton, S. (2012). Signaling network crosstalk in human pluripotent cells: a Smad2/3-regulated switch that controls the balance between self-renewal and differentiation. Cell stem cell 10, 312-326.
Sokol, S. Y. (2011). Maintaining embryonic stem cell pluripotency with Wnt signaling. Development 138, 4341-4350.
ten Berge, D., Kurek, D., Blauwkamp, T., Koole, W., Maas, A., Eroglu, E., Siu, R. K., and Nusse, R. (2011). Embryonic stem cells require Wnt proteins to prevent differentiation to epiblast stem cells. Nature cell biology 13, 1070-1075.
Tesar, P. J., Chenoweth, J. G., Brook, F. A., Davies, T. J., Evans, E. P., Mack, D. L., Gardner, R. L., and McKay, R. D. (2007). New cell lines from mouse epiblast share defining features with human embryonic stem cells. Nature 448, 196-199.
Thorne, C. A., Hanson, A. J., Schneider, J., Tahinci, E., Orton, D., Cselenyi, C. S., Jernigan, K. K., Meyers, K. C., Hang, B. I., Waterson, A. G., et al. (2010). Small-molecule inhibition of Wnt signaling through activation of casein kinase 1 alpha. Nature chemical biology 6, 829-836.
Tong, C., Huang, G., Ashton, C., Li, P., and Ying, Q. L. (2011). Generating gene knockout rats by homologous recombination in embryonic stem cells. Nat Protoc 6, 827-844.
van Amerongen, R., and Nusse, R. (2009). Towards an integrated view of Wnt signaling in development. Development 136, 3205-3214.
Wray, J., Kalkan, T., Gomez-Lopez, S., Eckardt, D., Cook, A., Kemler, R., and Smith, A. (2011) Inhibition of glycogen synthase kinase-3 alleviates Tcf3 repression of the pluripotency network and increases embryonic stem cell resistance to differentiation. Nature cell biology 13, 838-845.
Wray, J., Kalkan, T., and Smith, A. G. (2010). The ground state of pluripotency. Biochemical Society transactions 38, 1027-1032.
Yamada, S., Pokutta, S., Drees, F., Weis, W. I., and Nelson, W. J. (2005). Deconstructing the cadherin-catenin-actin complex. Cell 123, 889-901.
Yeom, Y. I., Fuhrmann, G., Ovitt, C. E., Brehm, A., Ohbo, K., Gross, M., Hubner, K., and Scholer, H. R. (1996). Germline regulatory element of Oct-4 specific for the totipotent cycle of embryonal cells. Development 122, 881-894.
Ying, Q. L., Wray, J., Nichols, J., Batlle-Morera, L., Doble, B., Woodgett, J., Cohen, P., and Smith, A. (2008). The ground state of embryonic stem cell self-renewal. Nature 453, 519-523.

### Supplemental Experimental Procedures

### Derivation and Propagation of EpiSCs

Post-implantation epiblasts were isolated from E5.75 embryos of CD1 (Charles River) and 129SvE (Taconic) mice, as previously described (Chenoweth and Tesar, 2010). Each epiblast was transferred to one drop (25 μl) of Cell Dissociation Buffer (Gibco) and incubated at room temperature for 3-5 minutes, after which the Reichert's membrane and visceral endoderm were surgically removed, using sharp glass needles. Each epiblast fragment was then placed into an individual well of a 4-well plate pre-coated with 0.1% gelatin. Epiblasts were cultured in either the FGF2/activin or the CHIR/XAV conditions. After 3-4 days, the epiblast outgrowths were disaggregated into small clumps and replated in the same conditions. Emerging EpiSCs were trypsinized and expanded every 2-3 days at a subculture ratio of 1:4. For derivation of rat EpiSCs, post-implantation epiblasts were isolated from E7.5 Sprague-Dawley and Dark Agouti rat embryos (Harlan) and cultured on MEFs in the CHIR/XAV condition. Animal experiments were performed according to the investigator's protocols approved by the University of Southern California Institutional Animal Care and Use Committee.

### Human ESC Culture

H1, H9, HES-2 and HES-3 human ESC lines were kindly provided by the University of Southern California Stem Cell Core Facility. Human ESCs were routinely maintained on γ-irradiated MEF feeders in Knockout™ DMEM medium (Invitrogen) supplemented with 20% Knockout™ serum replacement (KSR; Invitrogen), 10 ng/ml FGF2 (PeproTech), 1% nonessential amino acids, 2 mM L-glutamine, and 0.1 mM β-mercaptoethanol. For culture in the CHIR/IWR-1 or CHIR/XAV condition, human ESCs were plated onto dishes pre-coated with Matrigel™ (BD Biosciences) or pre-seeded with MEFs and cultured in DMEM/KSR or MEF-conditioned media supplemented with 3 μM CHIR99021, 2.5 μM IWR1, or 2 μM XAV939. MEF-conditioned medium was prepared as described (Xu et al., 2001). For passaging, human ESCs were dissociated into single cells with 0.05% trypsin or small clumps with the Calcium Trypsin KSR(CTK) solution every 2-4 days as previously described (Hasegawa et al., 2006), and replated into the CHIR/IWR-1 or CHIR/XAV condition.

To evaluate the colony-forming efficiency of human ESCs cultured in the FGF2 or the CHIR-IWR-1 condition, cells were trypsinized and passed through 40 μm cell strainer (BD Biosciences) to obtain single-cell suspension. Cells were then counted and seeded at a density of 1000 cells/well onto 6-well plates pre-seeded with MEFs. After 7 days, cells were fixed with 4% paraformaldehyde (PFA) and stained for alkaline phosphatase (AP) using the Vector Blue™ Substrate kit (Vector laboratories). Colony-forming efficiencies were calculated as the number of AP positive colonies formed divided by the number of cells plated.

### Generation of β-Catenin^{-/-} Mouse EpiSCs and ESCs

β-catenin^{fl/fl} EpiSCs were derived from B6.129-Ctnnb1^{tm2Kem}/KnwJ mice (The Jackson Laboratory) that possess loxP sites located in introns 1 and 6 of the Ctnnb 1 (6-catenin) gene (Brault et al., 2001) (FIG. 5A)., β-catenin^{fl/fl} EpiSCs were derived and maintained in the FGF2/activin condition. β-catenin^{-/-} ESCs were generated from β-catenin^{fl/fl} ESCs by transient transfection of the pCAG-Cre-IRES-Puro plasmid using Lipofectamine™ (Invitrogen). Transfectants were selected for 7 days in GMEM/10% FBS medium supplemented with 10 ng/ml LIF, 1 μM PD0325901, and 1 μg/ml puromycin. Puromycin-resistant ESC colonies were picked and expanded in the LIF+PD0325901 condition (LIF alone was not sufficient to maintain self-renewal of β-catenin^{-/-} ESCs). Loss of β-catenin in β-catenin^{-/-} ESCs was confirmed by Western blot analysis. β-catenin^{-/-} EpiSCs were generated from β-catenin^{fl/fl} EpiSCs by transient transfection of the pCAG-Cre-IRES-Puro plasmid or from β-catenin^{-/-} ESCs by culturing them in FGF2/activin condition (Guo et al., 2009; Hanna et al., 2009). β-catenin^{-/-} EpiSCs were routinely maintained in the FGF2/activin condition.

### Construction of 13-Catenin Mutant Plasmids.

pcDNA3-human β-catenin and pcDNA3-human ΔNβ-catenin plasmids (Kolligs et al., 1999) (Addgene) were double-digested with BamHI and NotI. Full-length and ΔNβ-catenin fragments were collected and ligated into the pCAG-IRES-hygro vector. The A295W and I296W point mutations (Graham et al., 2000) were introduced into full-length β-catenin and the ΔNβ-catenin mutant by PCR-driven overlap extension (Heckman and Pease, 2007) using the two PCR primer pairs described below. Floxed ΔNβ-catenin-ERT2 plasmid was constructed by insertion of the ΔNβ-catenin-ERT2 cassette into the pCAG-loxP-IRES-pac-STOP-loxP-EGFP-pA vector (Chambers et al., 2003). Full-length β-catenin or β-catenin mutants were transfected into mouse ESCs, mouse EpiSCs, and human ESCs by electroporation. Drug-resistant colonies were picked and expanded to establish stable cell lines.

Construction of Axin Expression Plasmids and Axin shRNA

Mouse Axin1 and Axin2 open reading frames (ORFs) were amplified by PCR from CD1 EpiSCs using KOD Hot Start DNA Polymerase (EMD). Axin1 and Axin2 ORFs were then cloned into the PiggyBac™ transposon vector and verified by DNA sequencing. For RNA interference of Axin1 and Axin2 in EpiSCs, short hairpin (shRNA) constructs designed to target 21 base-pair gene-specific regions in Axin1 and Axin2 were cloned into pLKO.1-TRC vector (Addgene). The targeted sequences were as follows:

(SEQ ID NO: 17)
Axin1, GCCACAGAAATTTGCTGAAGA;
(SEQ ID NO: 18)
Axin2, GGTTTGCTTGTAATGGGTTCA.

For overexpression of Axin1 or Axin2 in EpiSCs, 2 μg transposon vector was co-transfected with 2 μg PiggyBac™-Axin1 or PiggyBac™-Axin2 into EpiSCs using Lipofectamine® LTX & Plus reagent (Invitrogen) according to the manufacturer's instructions. 24 h after transfection, 1 μg/ml puromycin was added to the cell culture medium to select for transfected colonies. For RNAi experiments, pLKO.1-TRC-based lentiviral vectors were transfected with packaging plasmids pMD2.G and psPAX2 into 293FT cells (Invitrogen) using Lipofectamine® LTX & Plus reagent. Virus-containing supernatant was collected 48 h after transfection. EpiSCs were incubated in the virus supernatant supplemented with 8 μg/ml polybrene (Sigma) for 24 h. Supernatant was then replaced with fresh CHIR/IWR-1 medium supplemented with 1 μg/ml puromycin to select for transfected cells.

qRT-PCR

Total RNA was extracted with the RNeasy™ Mini Kit (Qiagen). cDNA was synthesized with 1 ng of total RNA, using the QuantiTech™ Rev. Transcription Kit (Qiagen). qRT-PCR was performed with Power SYBR™ Green PCR Master Mix (Applied Biosystems) according to the manufacturer's instructions. Signals were detected with an ABI7900HT Real-Time PCR System (Applied Biosystems). The relative expression level was determined by the 2-ACT method and normalized against GAPDH. The primers used for qRT-PCR are described below.

### Teratoma Formation and In Vitro Differentiation of Mouse EpiSCs and Human ESCs

Mouse EpiSCs and human ESCs maintained in CHIR/XAV or CHIR/IWR1 conditions were tested for their ability to form teratomas in immunodeficient SCID mice. Colonies were dissociated into small cell clumps with CTK solution and cells were resuspended in PBS at a concentration of 1×10⁷ cells/ml. Five hundred microliters of cell suspension was subcutaneously injected into right and left flank of 12 weeks old NOD SCID mice (Charles River). Tumors were allowed to develop for 8 weeks. Teratomas were removed and fixed in 4% paraformaldehyde for 48 hours, followed by paraffin embedding, sectioning, and staining with hematoxylin and eosin (H&E). In vitro EpiSC differentiation was induced by formation of embryoid bodies (EBs). EpiSC-derived EBs were plated onto gelatin-coated dishes and cultured in GMEM/10% FBS medium. Spontaneously beating cardiomyocytes appeared after 2 weeks in culture. Neural differentiation of EpiSCs was induced as previously described (Ying and Smith, 2003; Ying et al., 2003).

### Primer Sets for Generating 13Catenin A295W/I296W Point Mutation

Leading Fragment:
(SEQ ID NO: 19)
5′-ATAACGCGTCCAGCGTGGCAATGGCTCGA-3′;
(SEQ ID NO: 20)
5′-TGTCGTCCACCACAAGAATTTAACATTTGTTTT-3′
Following Fragment:
(SEQ ID NO: 21)
5′-TTCTTGTGGTGGACGACAGACTGCCTTCAAATT-3′;
(SEQ ID NO: 22)
5′-ATAGCGGCCGCTTACTTGTCATCGTCGTCCT-3′
Overlap extension:
(SEQ ID NO: 23)
5′-ATAACGCGTCCAGCGTGGCAATGGCTCGA-3′;
(SEQ ID NO: 24)
5′-ATAGCGGCCGCTTACTTGTCATCGTCGTCCT-3′

Primer Pairs for qRT-PCR

Oct4:
(SEQ ID NO: 25)
5′-GAAGCAGAAGAGGATCACCTTG-3′;
(SEQ ID NO: 26)
5′-TTCTTAAGGCTGAGCTGCAAG-3′
Rex1:
(SEQ ID NO: 27)
5′-TCACTGTGCTGCCTCCAAGT-3′;
(SEQ ID NO: 28)
5′-GGGCACTGATCCGCAAAC-3′
Nr0b1:
(SEQ ID NO: 29)
5′-TCCAGGCCATCAAGAGTTTC-3′;
(SEQ ID NO: 30)
5′-ATCTGCTGGGTTCTCCACTG-3′
Fgf5:
(SEQ ID NO: 31)
5′-GCAGCCCACGGGTCAA-3′;
(SEQ ID NO: 32)
5′-CGGTTGCTCGGACTGCTT-3′
Stella:
(SEQ ID NO: 33)
5′-TTCCGAGCTAGCTTTTGAGG-3′;
(SEQ ID NO: 34)
5′-ACACCGGGGTTTAGGGTTAG-3′
Gapdh:
(SEQ ID NO: 35)
5′-TGAAGCAGGCATCTGAGGG-3′;
(SEQ ID NO: 36)
5′-CGAAGGTGGAAGAGTGGGAG-3′
Nanog:
(SEQ ID NO: 37)
5′-TCCAGAAGAGGGCGTCAGAT-3′;
(SEQ ID NO: 38)
5′-CAAATCCCAGCAACCACATG-3′
Axin1:
(SEQ ID NO: 39)
5′-TTAGGTGTCTGCCAGCCTCT-3′;
(SEQ ID NO: 40)
5′-AACCAGGTGCAGTGGATAGG-3′
Axin2:
(SEQ ID NO: 41)
5′-GGGGGAAAACACAGCTTACA-3′;
(SEQ ID NO: 42)
5′-TTGACTGGGTCGCTTCTCTT-3′

### SUPPLEMENTAL REFERENCES

Brault, V., Moore, R., Kutsch, S., Ishibashi, M., Rowitch, D. H., McMahon, A. P., Sommer, L., Boussadia, O., and Kemler, R. (2001). Inactivation of the beta-catenin gene by Wnt1-Cre-mediated deletion results in dramatic brain malformation and failure of craniofacial development. Development 128, 1253-1264.
Chambers, I., Colby, D., Robertson, M., Nichols, J., Lee, S., Tweedie, S., and Smith, A. (2003). Functional expression cloning of Nanog, a pluripotency sustaining factor in embryonic stem cells. Cell 113, 643-655.
Chenoweth, J. G., and Tesar, P. J. (2010). Isolation and maintenance of mouse epiblast stem cells. Methods Mol Biol 636, 25-44.
Graham, T. A., Weaver, C., Mao, F., Kimelman, D., and Xu, W. (2000). Crystal structure of a beta-catenin/Tcf complex. Cell 103, 885-896.
Guo, G., Yang, J., Nichols, J., Hall, J. S., Eyres, I., Mansfield, W., and Smith, A. (2009). Klf4 reverts developmentally programmed restriction of ground state pluripotency. Development 136, 1063-1069.
Hanna, J., Markoulaki, S., Mitalipova, M., Cheng, A. W., Cassady, J. P., Staerk, J., Carey, B. W., Lengner, C. J., Foreman, R., Love, J., et al. (2009). Metastable pluripotent states in NOD-mouse-derived ESCs. Cell stem cell 4, 513-524.
Hasegawa, K., Fujioka, T., Nakamura, Y., Nakatsuji, N., and Suemori, H. (2006). A method for the selection of human embryonic stem cell sublines with high replating efficiency after single-cell dissociation. Stem cells 24, 2649-2660.
Heckman, K. L., and Pease, L. R. (2007). Gene splicing and mutagenesis by PCR-driven overlap extension. Nat Protoc 2, 924-932.

Kolligs, F. T., Hu, G., Dang, C. V., and Fearon, E. R. (1999). Neoplastic transformation of RK3E by mutant beta-catenin requires deregulation of Tcf/Lef transcription but not activation of c-myc expression. Mol Cell Biol 19, 5696-5706. Xu, C., Inokuma, M. S., Denham, J., Golds, K., Kundu, P., Gold, J. D., and Carpenter, M. K. (2001). Feeder-free growth of undifferentiated human embryonic stem cells. Nat Biotechnol 19, 971-974.
Ying, Q. L., and Smith, A. G. (2003). Defined conditions for neural commitment and differentiation. Methods Enzymol 365, 327-341.
Ying, Q. L., Stpyridis, M., Griffiths, D., Li, M., and Smith, A. (2003). Conversion of embryonic stem cells into neuroectodermal precursors in adherent monoculture. Nat Biotechnol 21, 183-186.

### Example 2

### 53AH Data

53AH is a selective Wnt pathway inhibitor. It is a cyclohexyl analog of IWR-1 with defined centers of chirality¹. Compared to IWR-1, 53AH has 5-fold greater potency in Wnt inhibition¹. CD1 mouse EpiSCs were cultured in GMEM/10% FBS medium supplemented with 3 μm CHIR99021 and 1 μM 53AH. FIG. 10 shows CD1 EpiSCs after 21 passages in CHIR/53AH.

H9 human ESCs were plated onto Matrigel™-coated dishes and cultured in serum-free N2B27 only. They differentiated after 7 days in culture (FIG. 11A). H9 human ESCs plated onto Matrigel™-coated dishes and cultured in serum-free N2B27 supplemented with 3 μM CHIR99021 and 1 μM 53AH were maintained in this condition for over 10 passages and still remain undifferentiated (FIG. 11B).

These studies showed that 53AH is more robust for long-term expansion of mouse EpiSCs and human ESCs compared to IWR-1, XAV939, and JW55.

### Example 3

### Chicken ES Stem Cell Data

We isolated blastodermal cells from stage X embryos of the fertile Rhode Island Red brown eggs according the protocol described by van de Lavoir². These cells were plated onto MEF-coated 4-well plates and cultured in N2B27 medium supplemented with 3 μM CHIR99021 and 1 μM 53AH. ES-like cells can be maintained under this condition for up to 5 passages (FIG. 3).

### REFERENCES FOR EXAMPLES 2-3

1. Willems E, et al, Small-molecule inhibitors of the Wnt pathway potently promote cardiomyocytes from human embryonic stem cell-derived mesoderm. Circ Res. 2011; 109(4):3604. PMID: 21737789.
2. van de Lavoir M C, Mather-Love C. Avian embryonic stem cells. Methods Enzymol. 2006; 418:38-64.

We claim:

1. A composition, comprising
(a) an inhibitor of β-catenin binding to T-cell factors (Tcfs); and
(b) a suppressor of glycogen synthase kinase (GSK3) activation.

2. The composition of claim 1, wherein the inhibitor of β-catenin binding to Tcfs is selected from the group consisting of 3,5,7,8-Tetrahydro-2-[4-(trifluoromethyl)phenyl]-4H-thiopyrano[4,3-d]pyrimidin-4-one (XAV939), 4-(1,3,3a,4,7,7a-Hexahydro-1,3-dioxo-4,7-methano-2H-isoindol-2-yl)-N-8-quinolinyl-Benzamide (IWR-1), and (1R,4r)-4-((2s,3aR,4R,7S,7aS)-1,3-dioxooctahydro-1H-4,7-methanoinden-2-yl)-N-(quinolin-8-yl)cyclohexanecarboxamide (53AH), or salts thereof.

3. The composition of claim 1, wherein the suppressor of GSK3 activation is selected from the group consisting of 6-((2-((4-(2,4-Dichlorophenyl)-5-(4-methyl-1H-imidazol-2-yl)pyrimidin-2-yl)amino)ethyl)amino)nicotinonitrile (CHIR99021), 2,6-Pyridinediamine, N6-[2-[[4-(2,4-dichlorophenyl)-5-(1H-imidazol-1-yl)-2-pyrimidinyl]amino]ethyl]-3-nitro- (CHIR 98014), benzyl-2-methyl-1,2,4-thiadiazolidine-3,5-dione (TDZD-8), 3-(2,4-Dichlorophenyl)-4-(1-methyl-1H-indol-3-yl)-1H-pyrrole-2,5-dione (SB216763), 3-[(3-Chloro-4-hydroxyphenyl)amino]-4-(2-nitrophenyl)-1H-pyrrol-2,5-dione (SB415286) HIR98014, Wnt3a, AR-AO144-18, or salts thereof.

4. The composition of claim 2, wherein the suppressor of GSK3 activation is selected from the group consisting of 6-((2-((4-(2,4-Dichlorophenyl)-5-(4-methyl-1H-imidazol-2-yl)pyrimidin-2-yl)amino)ethyl)amino)nicotinonitrile (CHIR99021), 2,6-Pyridinediamine, N6-[2-[[4-(2,4-dichlorophenyl)-5-(1H-imidazol-1-yl)-2-pyrimidinyl]amino]ethyl]-3-nitro- (CHIR 98014), benzyl-2-methyl-1,2,4-thiadiazolidine-3,5-dione (TDZD-8), 3-(2,4-Dichlorophenyl)-4-(1-methyl-1H-indol-3-yl)-1H-pyrrole-2,5-dione (SB216763), 3-[(3-Chloro-4-hydroxyphenyl)amino]-4-(2-nitrophenyl)-1H-pyrrol-2,5-dione (SB415286); 2,6-Pyridinediamine, N6-[2-[[4-(2,4-dichlorophenyl)-5-(1H-imidazol-1-yl)-2-pyrimidinyl]amino]ethyl]-3-nitro-; N-[(4-Methoxyphenyl)methyl]-N′-(5-nitro-2-thiazolyl); and Wnt3a (SEQ ID NO: 15 or 16), or salts thereof.

5. The composition of claim 4, wherein the inhibitor of β-catenin binding to Tcfs is (1R,4r)-4-((2s,3aR,4R,7S,7aS)-1,3-dioxooctahydro-1H-4,7-methanoinden-2-yl)-N-(quinolin-8-yl)cyclohexanecarboxamide or a salt thereof, and the suppressor of GSK3 activation is 6-((2-((4-(2,4-Dichlorophenyl)-5-(4-methyl-1H-imidazol-2-yl)pyrimidin-2-yl)amino)ethyl)amino)nicotinonitrile or a salt thereof.

6. The composition of claim 1, further comprising
(c) basal cell culture medium,
wherein the composition comprises a cell culture medium

7. The composition of claim 6, further comprising
(c) basal cell culture medium,
wherein the composition comprises a cell culture medium.

8. The cell culture medium of claim 7, wherein the (1R,4r)-4-((2s,3aR,4R,7S,7aS)-1,3-dioxooctahydro-1H-4,7-methanoinden-2-yl)-N-(quinolin-8-yl)cyclohexanecarboxamide or salt thereof is present in the cell culture medium at a concentration of between about 1 μM and about 10 μM.

9. The cell culture medium of claim 7, wherein the 6-((2-((4-(2,4-Dichlorophenyl)-5-(4-methyl-1H-imidazol-2-yl)pyrimidin-2-yl)amino)ethyl)amino)nicotinonitrile or salt thereof is present in the cell culture medium at a concentration of between about 1 μM and about 10 μM.

10. The cell culture medium of claim 8, wherein the 6-((2-((4-(2,4-Dichlorophenyl)-5-(4-methyl-1H-imidazol-2-yl)pyrimidin-2-yl)amino)ethyl)amino)nicotinonitrile or salt thereof is present in the cell culture medium at a concentration of between about 1 μM and about 10 μM.

11. The cell culture medium of claim 10, wherein the (1R,4r)-4-((2s,3aR,4R,7S,7aS)-1,3-dioxooctahydro-1H-4,7-methanoinden-2-yl)-N-(quinolin-8-yl)cyclohexanecarboxamide or salt thereof, and the 6-((2-((4-(2,4-Dichlorophenyl)-5-(4-methyl-1H-imidazol-2-yl)pyrimidin-2-yl)amino)ethyl)amino)nicotinonitrile or salt thereof are present in the cell culture medium at a ratio of between about 1:3 and 3:1.

12. A method for culturing pluripotent stem cells, comprising culturing the pluripotent stem cells in the culture medium of claim 6 under conditions suitable for culturing the pluripotent stem cells.

13. The method of claim 12, wherein the pluripotent stem cells comprise embryonic stem cells (ESCs) or epiblast-derived stem cells (EpiSCs).

14. The method of claim 12, wherein the pluripotent stem cells are from an organism selected from the group consisting of mice, rats, cows, rabbits, pigs, humans, and chickens.

15. A method for generating a pluripotent cell line from a tissue, comprising
(a) culturing a tissue comprising a pluripotent cell in the cell culture medium of claim 6; and
(b) isolating the pluripotent cells in the culture medium.

16. The method of claim 15, wherein the tissue is selected from the group consisting of blastocysts, fertilized embryos, inner cell mass (ICM) tissue, or adult tissue.

17. Isolated pluripotent cells isolated by the method of claim 15.
